(19) United States
(12) Patent Application Publication
(10) Pub. No.: US 2016/0213766 A1
(43) Pub. Date: Jul. 28, 2016

(54) OspA Fusion Protein for Vaccination against Lyme Disease

(71) Applicant: VENTRIA BIOSCIENCE, INC., Fort Collins, CO (US)

(72) Inventors: Ning HUANG, Davis, CA (US); Deshui ZHANG, Woodland, CA (US); Barbara J.B. JOHNSON, Fort Collins, CO (US); Chris MACMANUS, N/A (IL)

(73) Assignee: Ventria Bioscience, Inc., Fort Collins, CO (US)

(21) Appl. No.: 15/024,036

(22) PCT Filed: Sep. 23, 2014
(86) PCT No.: PCT/US14/00191

§ 371 Date: Mar. 23, 2016

Related U.S. Application Data
(60) Provisional application No. 61/881,387, filed on Sep. 23, 2013.

(51) Int. Cl. A61K 39/02; A61K 39/39; A61K 45/06; C07K 14/20; C07K 14/28

(52) U.S. Cl. CPC A61K 39/0225; C07K 14/20; C07K 14/28; A61K 45/06; A61K 39/39; C07K 2319/55; C07K 2319/40; A61K 2039/55544

(57) ABSTRACT

Provided herein are monocot seed compositions and methods of making a monocot seed product expressing high levels of recombinant Osp fusion protein. In some embodiments, a rice seed composition is used in the manufacture of a Lyme disease vaccine formulation. In some embodiments, the composition comprising the Osp fusion protein is admixed with a drug or pharmacologically active agent, such as an antibiotic.

### CROSS REFERENCE TO RELATED APPLICATIONS

This application claims benefit under 35 U.S.C. §119(e) to U.S. Provisional Patent Application Ser. No. 61/881,387, filed 23 Sep. 2013, the contents of which are incorporated herein by reference.

### STATEMENT REGARDING GOVERNMENT INTEREST

This invention was made with Government support under contract AI081339 awarded by the National Institutes of Health. The Government has certain rights in this invention.

### REFERENCE TO SEQUENCE LISTING, TABLE OR COMPUTER PROGRAM

A Sequence Listing is being submitted electronically via EFS in the form of a text file, created 22 Sep. 2014, and named “VBI8040W000SeqList.txt” (12,288 bytes), the contents of which are incorporated herein by reference in their entirety.

61/881,387

### TECHNICAL FIELD

The present disclosure relates, generally, to compositions comprising a fusion protein of an adjuvant to a Borrelia outer surface protein antigen such as the Outer Surface Protein A (OspA), and to methods for recombinantly producing one or more Osp proteins in monocot plants, monocot plant cells and seeds, which may be used, for example, in the development and generation of an Osp-specific vaccine for orally vaccinating an animal against Lyme disease, or for preventing, ameliorating and/or treating infection of an animal by Borrelia species. The present disclosure further relates to recombinantly-produced adjuvant-Borrelia antigen fusion protein(s) provided in a form suitable for oral administration as a vaccine, in order to prevent an animal from acquiring infection with a Lyme disease pathogen after subsequent exposure to a source of Borrelia burgdorferi.

### BACKGROUND

A zoonosis is an infectious disease transmitted between species (sometimes by a vector) from non-human animals to humans; when the transmission occurs from humans to other animals it is called “reverse zoonosis” or “anthroponosis.” In a systematic review of 1,415 pathogens known to infect humans. 61% were found to be zoonotic. Zoonoses can be classified according to the following infectious agent types: Parasites (e.g., protozoa and helminths such as nematodes, cestodes and trematodes) fungi, bacteria, viruses, prions.

A partial list of vectors known to carry zoonotic infectious organisms is as follows: apes (e.g., chimpanzee, gorilla), monkeys (e.g., macaques), assassin bugs, mosquitos, fleas, flies, bats, bank voles, birds, cats, cattle, copepods, dogs, fish, foxes, geese, goats, hamsters, horses, hyraxes, lice, opossums, raccoons, pigs, rabbits and hares, rodents (e.g., mice, rats), sloths, sheep, snails, ticks and wolves. In some cases, a vector may also be referred to as a “reservoir” or “reservoir animal.”

Examples of zoonoses are: anthrax, babesiosis, balantidiasis, barmah Forest virus, bartonellosis, bilharzia, Bolivian hemorrhagic fever, brucellosis, borreliosis (e.g., Lyme disease and others), borna virus infection, bovine tuberculosis, campylobacteriosis, cat scratch disease, Chagas disease, cholera, cowpox Creutzfeldt-Jakob disease (vCJD), transmissible spongiform encephalopathy (TSE), bovine spongiform encephalopathy (BSE) or “mad cow disease,” Crimean-Congo hemorrhagic fever (CCHF), cryptosporidiosis, cutaneous larva migrans, dengue fever, Ebola, echinococcosis, Escherichia coli O157:H7, erysipeloid, eastern equine encephalitis virus, western equine encephalitis virus, Venezuelan equine encephalitis virus, giardiasis, glanders, H1N1 flu, hantavirus, helminths, Hendra virus, Henipavirus, Human Immunodeficiency Virus (HIV), Korean hemorrhagic fever, Kyasanur forest disease, Lábrea fever, Lassa fever, leishmaniasis, leptospirosis, listeriosis, lymphocytic choriomeningitis virus, Marburg fever, mediterranean spotted fever, Mycobacterium marinum, Monkey B, Nipah fever, ocular larva migrans, Omsk hemorrhagic fever, ornithosis, Orf (animal disease), Oropouche fever, pappataci fever, pasteurellosis, plague, psittacosis, Puumala virus, Q-Fever, rabies, Rift Valley fever, ringworms (Tinea canis), salmonellosis, SARS, sodoku, sparganosis, Streptococcus suis, toxocariasis, toxoplasmosis, trichinosis, tularemia, typhus of Rickettsiae, Venezuelan hemorrhagic fever, Visceral larva migrans, West Nile virus, yellow fever and yersiniosis.

Lyme disease (or borreliosis) is a zoonotic, vector-borne disease caused by a spirochetal bacterium from the genus Borrelia, and transmitted to humans by the bite of infected Ixodes ticks. The life cycle of Borellia burgdorferi is complex, and may require tick, rodent, and deer hosts at various points. Rodents are the primary reservoir for the bacterium; the white-footed mouse is one reservoir for the maintenance of B. burgdorferi. Deer ticks (Ixodes scapularis) then feed on these mouse populations, and thereby transmit the B. burgdorferi infection to deer. Then, in endemic areas, the ticks also feed on humans, and in doing so, spread B. burgdorferi to people.

The number of reported cases of Lyme disease has been steadily increasing worldwide, and it is one of the fastest-growing infectious diseases in the United States; thus it is a public health imperative to control its spread, as it has proven difficult to diagnose and treat. Current measures to prevent B. burgdorferi infections in humans have been wholly inadequate. Below, the detailed description of present disclosure solves this problem by providing a vaccine for the successful reduction in the rate of B. burgdorferi infection and/or prevention of transmission of Lyme disease to humans, or any other mammal in the wild. The presently described vaccine incorporates an antigen from B. burgdorferi into a bait for rodents, which is then fed to wild mice in residential areas where most human infections occur. Thus, the mice are protected from B. burgdorferi infection, and further, when infected ticks feed on the immunized mice, the ticks, too, become cleared of B. burgdorferi. This two-fold mode of action of an oral vaccine reduces the risk of Lyme disease in people and other animals in areas where the vaccine is used.

The outer membrane of Borrelia burgdorferi is composed of various unique outer surface proteins (Osp) characterized as OspA through OspF. The Osp proteins are lipoproteins anchored to the membrane by N-terminally attached fatty acid molecules, and they are presumed to play a role in virulence, transmission, or survival of the bacterium in the tick (Haake, (2000) Microbiology 146(7):1491-1504). OspA, OspB, and OspD are expressed by B. burgdorferi bacteria residing in the gut of unfed ticks, and it has been suggested these lipoproteins promote the persistence of the spirochete in ticks between blood meals (Schwan, et al., (1995) Proc. Natl. Acad. Sci. USA 92:2909-2913). The OspA and OspB genes, which have a high degree of sequence similarity, encode the major outer membrane proteins of B. burgdorferi. Virtually all spirochetes in the midgut of an unfed nymph tick express OspA. OspA promotes the attachment of B. burgdorferi to the tick protein TROSPA, present on tick gut epithelial cells. OspB also has an essential role in the adherence of B. burgdorferi to the tick gut.

U.S. Pat. No. 6,183,986 (Bergstrom et al.) describes the isolation and sequencing of the outer surface protein A (OspA) gene of B. burgdorferi, as well as using an immunogenic fragment expressed from a viral vector in a vaccine. OspA expressed by Borrelia burgdorferi in the tick mid-gut has been used as an antigen; humans and mice vaccinated with OspA protein were protected from B. burgdorferi infection. (See Steere et al., N Engl. J. Med. 339:209-15 (1998)). A vaccine based on OspA of Borrelia, called Lymerix, has also been developed to control Lyme Disease in humans. (See Sigal et al., N Engl. J Med. 339:216-2 (1998)). However, the Lymerix vaccine was pulled from the market in 2002 for numerous reasons including high expense, poor market conditions and safety concerns.

Gomes-Solecki et al. have described an oral bait delivery system containing an OspA protein obtained from transformed E. coli. OspA expressed in E. coli was found to be immunogenic when administered by injection or orally. In fact, the E. coli expressed OspA acted as an oral vaccine, protecting 89% of mice from infection, and resulted in an eight-fold reduction in the amount of B. burgdorferi present in tick vectors (Vaccine 24:4440-49 (2006)). However, E. coli expression of OspA is costly and requires precautions to prevent release of live E. coli into the environment.

Attempts to express OspA in plants were thwarted by the observation that OspA was toxic when expressed in leaves of tobacco plants. Hennig et al. describe successful transformation of tobacco plant leaf cells and expression of recombinant OspA protein in chloroplasts using a signal peptide from OspA. However, those transgenic plants accumulating OspA in higher amounts (>1% total soluble protein (TSP)) had a plant cell metabolic disorder, and were incapable of carrying out sufficient photosynthesis. Thus, Hennig et al. found that expression of OspA tobacco plant leaf cells was toxic, could not grow without exogenously supplied sugars and rapidly died after transfer to soil under greenhouse conditions unless sugars were exogenously applied. (FEBS J 274(21):5749-5758 (2007)).

Accordingly, there remains an unmet need in the art for high-level expression of recombinant Osp proteins in plants, where the growth of the plant is not compromised, so as to be able to produce large amounts of the proteins over multiple generations.

US Patent Application Publication 20110117131 (Huang, et al.), incorporated by reference herein in its entirety, describes the generation of transgenic rice expressing recombinant OspA (rOspA) protein for the use as a vaccine. The compositions and methods described therein provide successfully transformed transgenic monocots that grew to maturity, were fertile and produced seeds expressing Outer surface protein A (OspA) as at least 2% of the total soluble protein in the seed in a phenotypically normal transgenic monocot plant. By using this monocot seed expression system which targets expression of the protein to plant seeds, the growth, photosynthetic ability and fertility of the plant is not compromised. Thus, OspA was expressed at high levels in seeds rather than leaves, avoiding the metabolic toxicity problem observed in tobacco. This monocot seed expression system provides transgenic plants able to produce large amounts of the seed-expressed OspA protein over multiple generations. Furthermore, this plant-expressed OspA protein was immunogenic when administered by injection. However, to date, oral administration of this plant-expressed OspA failed to generate protective antibodies or to protect mice from infection by B. burgdorferi, even when mixed with a mucosal adjuvant. (It should be noted, as an aside, that even when infected by B. burgdorferi, rodents to not suffer from Lyme disease).

Another problem in the art is that very few proteins that are ingested have the ability to act as a vaccine. Thus, a strategy is required for effectively presenting ingested recombinant Osp proteins in a way that will trigger a protective response against B. burgdorferi. Clearly, it would be advantageous to produce large amounts of recombinant Osp protein suitable for use as an oral vaccine, and production of such a protein in plants is highly desirable, because plant protein production avoids contamination by human pathogens and is cost effective, particularly when yields are high. Such plant-produced recombinant Osp proteins are suitable for inclusion in compositions for orally vaccinating an animal host, to break the transmission cycle of Lyme disease. Accordingly, there is a need in the art for high-level expression of recombinant Osp proteins in plants, where the growth of the plant is not compromised, so as to be able to produce large amounts of the proteins over multiple generations. Such Osp proteins should be suitable for inclusion in compositions for orally vaccinating an animal host, in order to break the transmission cycle of Lyme disease.

It is to be understood that the foregoing description of the related art and the limitations presented therein are intended to be illustrative and not exclusive. Other limitations of the related art will become apparent to those of skill in the art upon a reading of the instant specification and a study of the drawings.

### BRIEF SUMMARY

Provided herein is a plant-expressed fusion protein comprising cholera toxin B subunit (CTB) adjuvant fused to a Borrelia outer surface protein A (OspA) protein, polypeptide or peptide fragment thereof.

In one aspect of the present disclosure, a codon-optimized nucleic acid sequence having at least 90% sequence identity to SEQ ID NO: 1, encoding a cholera toxin B subunit (CTB) adjuvant fused to an outer surface protein A (OspA) protein, polypeptide or peptide fragment thereof, is provided. In one aspect, an amino acid sequence having at least 90% sequence identity to the sequence identified by (SEQ ID NO: 2) is provided. In one aspect, an amino acid sequence having at least 90% sequence identity to the sequence identified by (SEQ ID NO: 2) and encoded by the codon-optimized nucleic acid sequence having at least 90% sequence identity to SEQ ID NO: 1 is provided.

In some aspects, a transgenic monocot plant expressing a CTB.OspA fusion protein having at least 90% sequence identity to the sequence identified by SEQ ID NO: 2 is provided.

In one aspect, a chimeric gene for expression of a CTB.OspA fusion protein is provided, said chimeric gene comprising: (i) a glutelin promoter that is active in monocot plant cells; (ii) an optional first nucleic acid sequence, operably linked to the promoter, encoding a monocot plant seed-specific signal peptide; and (iii) a (second) nucleic acid sequence identified by SEQ ID NO: 1, operably linked to the promoter, encoding a CTB.OspA fusion protein. In one aspect, a rice seed product comprising the fusion protein expressed by the chimeric gene is provided.

In some aspects, a formulation for oral administration to an animal is provided, which comprises the CTB.OspA (synonymously referred to as “CTB-OspA”) fusion protein having at least 90% sequence identity to the sequence identified by SEQ ID NO: 2. In some embodiments, the formulation is administered orally in an amount effective to induce the production of specific antibodies to an OspA protein in the animal, wherein said antibodies are effective to ameliorate or clear infection by a Borrelia species pathogen in a mammal. In some embodiments, and depending on the subject/animal, the formulation is orally administered in an amount from about 1 mg to about 10 g of the at least one CTB.OspA fusion protein per day.

In some aspects, a method is provided for immunizing an animal against infection with a Borrelia species pathogen, comprising the step of administering a formulation comprising at least one CTB.OspA fusion protein, wherein the at least one CTB.OspA fusion protein is obtained by extraction from the rice seed product.

In some aspects, a method is provided for immunizing an animal against infection with a Borrelia species pathogen, comprising the step of administering a formulation comprising the rice seed product to the animal.

In some aspects, a method is provided for producing seeds that express a cholera toxin B subunit (CTB).OspA fusion protein, wherein the method comprises: (a) transforming a monocot plant cell with the chimeric gene described herein; (b) producing a plant from the transformed plant cell and growing it for a time sufficient to produce seeds containing the fusion protein; and (c) harvesting the seeds from the plant. In some embodiments, the plant of step (b) is fertile and phenotypically normal.

In some aspects, an oral vaccine composition is provided, which comprises at least one CTB.OspA fusion protein and one or more excipients formulated for oral administration.

In some aspects, an oral vaccine is produced by a) providing a transgenic plant cell expressing the chimeric gene described herein, b) producing a plant from the transgenic plant cell and growing it for a time sufficient to produce seeds containing the CTB.OspA fusion protein, c) harvesting mature seeds containing the CTB.OspA fusion protein, d) grinding the mature seeds into small particles, e) optionally purifying the CTB.OspA fusion protein from the seeds, f) optionally producing a flour from the mature seeds, and g) combining the CTB.OspA with one or more excipients.

In some embodiments, the formulation is provided in a form selected from the group consisting of a bait, pellet, tablet, caplet, hard capsule, soft capsule, lozenge, cachet, powder, granules, suspension, solution, elixir, liquid, beverage, and food.

In some aspects, a method is provided for breaking a Lyme disease cycle by controlling pathogen prevalence in reservoir animals, comprising the steps of: a) expressing a CTB.OspA fusion protein having at least 90% sequence identity to the sequence identified by SEQ ID NO: 2 in monocot seeds; b) producing a rice flour from the monocot seeds; c) formulating the rice flour into a reservoir-targeting oral vaccine formulation without extracting the CTB.OspA fusion protein; and d) administering the formulation to Lyme disease reservoirs to induce immunity in reservoir species, thus reducing pathogen levels in reservoir animals and associated vectors.

In some aspects, a method is provided for eliminating a Borrelia species pathogen from a tick vector, comprising the step of administering the formulation described herein to a host animal and allowing the tick vector to feed on the host animal.

In some aspects, a method is provided for producing a CTB.OspA fusion protein in monocot plants, comprising the steps of: (a) transforming a monocot plant cell with the chimeric gene described herein; (b) producing a monocot plant from the transformed monocot plant cell and growing it for a time sufficient to produce seeds containing the OspA; and (c) harvesting the seeds from the monocot plant. In some embodiments, the plant of step (b) is fertile and phenotypically normal. In some embodiments, the monocot plant is selected from the group consisting of rice, barley, wheat, oat, rye, corn, millet, triticale and sorghum. In some embodiments, the monocot plant is rice.

In some embodiments, the CTB.OspA fusion protein comprises about 2% or greater of the total soluble protein in the seeds. In some embodiments, the CTB.OspA fusion protein comprises about 3% or greater of the total soluble protein in the seeds.

In some aspects, a method is provided for modifying or converting a non-mucosal-active microbial antigen to a mucosally-active vaccine for use in the prevention, amelioration or treatment of infection by a microbial species, comprising the steps of (a) preparing an expression vector comprising a monocot seed storage protein promoter active in plant seed cells operably linked to a chimeric gene encoding a fusion protein consisting of an adjuvant protein and non-mucosally-active microbial antigen; (b) transforming monocot plant cells with the expression vector; (c) selecting transformed monocot plant cells harboring the chimeric gene; (d) growing a plant from the selected transformed plant cells for a time sufficient to produce seeds expressing the fusion protein; (e) immunizing animals with the fusion protein via a mucosal surface to generate a protective immune-response against the microbial species.

Additional embodiments of the present methods and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure and claims. Additional aspects and advantages of the present disclosure are set forth in the following description and claims, particularly when considered in conjunction with the accompanying examples and drawings.

### BRIEF DESCRIPTION OF THE FIGURES

FIG. 1 shows the average daily consumption of various feeding baits.

FIG. 2 is a Western blot showing the reactivity of serum from OspA immunized mice against whole cell B. burgdorferi antigens

FIGS. 3A through 3E show an annotated nucleotide sequence and restriction map of the VB52 construct.

FIG. 4 is an illustration of the VB53 Gt1-CTB-OspA fusion protein expression construct.

FIG. 5 is a dot blot of transgenic plant lines expressing CTB.OspA fusion protein.

FIG. 6 is a photograph of a field of transgenic plants expressing CTB.OspA fusion protein.

FIG. 7 is a Western blot quantifying the expressed, purified and concentrated CTB.OspA fusion protein.

FIG. 8 is a Western blot comparing the oligomeric organization of CTB.OspA and recombinant OspA proteins from rice seeds under native and denaturing conditions.

FIG. 9 graphs a GM1 binding assay.

FIG. 10 shows a dot blot analysis of CTB.OspA fusion proteins.

FIG. 11 presents serum LA-2 titers in mice orally immunized with rice-derived CTB.OspA fusion protein.

### BRIEF DESCRIPTION OF THE SEQUENCES

SEQ ID NO: 1 presents a codon-optimized CTB.OspA fusion nucleotide sequence.

SEQ ID NO: 2 presents the amino acid sequence of a CTB.OspA fusion protein.

SEQ ID NO: 3 presents the nucleotide sequence of the VB53 plasmid construct.

### DETAILED DESCRIPTION

Oral immunization of a vaccine has several distinct advantages. For example, a vaccine which may be fed to subjects is significantly easier to administer on a large scale without the need for special equipment or needles, especially to subjects such as livestock and wild animals which may be difficult to handle or locate. An oral vaccine may be provided in the form of an edible solid, which is easier to handle under extreme conditions and is more stable than the liquid suspensions as currently used. Also, oral vaccination would eliminate infections spread by the re-use of needles. Moreover, delivery of immunogens to a mucosal membrane, such as by oral or intranasal vaccination, permits a secretory immune response to be raised. The secretory immune response, mainly IgA-mediated, is distinct from a systemic immune response, and systemic vaccination is ineffective for raising a secretory immune response. Thus, when considering immunization against pathogens which often enter the subject across a mucosal surface such as the gut or lung, delivery via a mucosal membrase has considerable advantages.

Current measures to prevent B. burgdorferi infections in humans are inadequate. The present disclosure provides a plant-expressed fusion protein comprising cholera toxin B subunit (CTB) adjuvant fused to a Borrelia outer surface protein A (OspA) protein, polypeptide or peptide fragment thereof. In one aspect of the present disclosure, a codon-optimized nucleic acid sequence having at least 90% sequence identity to SEQ ID NO: 1, encoding a cholera toxin B subunit (CTB) adjuvant fused to an outer surface protein A (OspA) protein, polypeptide or peptide fragment thereof, is provided. In one aspect, an amino acid sequence (SEQ ID NO: 2) encoded by the codon-optimized nucleic acid sequence having at least 90% sequence identity to SEQ ID NO: 1 is provided.

The present disclosure provides a vaccine for the successful reduction in the rate of B. burgdorferi infection and/or prevention of transmission of Lyme disease to humans, or any other mammal in the wild. This Lyme disease vaccine incorporates an antigen from B. burgdorferi into a bait for rodents, which is then fed to wild mice in residential areas where most human infections occur. Thus, the mice are protected from B. burgdorferi infection, and further, when infected ticks feed on the immunized mice, the ticks, too, become cleared of B. burgdorferi. This two-fold mode of action of an oral vaccine reduces the risk of Lyme disease in people and other animals in areas where the vaccine is used.

Very few proteins that are ingested have the ability to act as a vaccine. Therefore, a strategy was developed for enhancing the presentation of ingested recombinant OspA (rOspA) protein to immune cells for stimulating a protective response against B. burgdorferi. Adjuvants are a broad range of substances which display carrier and immunostimulatory properties, thereby increasing the efficacy of a vaccine by direct interaction and modulation of cells of the immune system. The ADP-ribosylating bacterial toxins, namely diphtheria toxin, pertussis toxin (PT), cholera toxin (CT), the E. coli heat-labile toxin (LT1 and LT2), Pseudomonas endotoxin A, C. botulinum C2 and C3 toxins as well as toxins from C. perfringens, C. spiriforma and C. difficile are potent toxins in man. These toxins are composed of a monomeric, enzymatically active A subunit which is responsible for ADP-ribosylation of GTP-binding proteins, and a non-toxic B subunit which binds receptors on the surface of the target cell and delivers the A subunit across the cell membrane. In the case of CT and LT, the A subunit is known to increase intracellular cAMP levels in target cells, while the B subunit is pentameric and binds to GM1 ganglioside receptors.

Other examples of mucosally active adjuvants are monophosphoryl lipid A (MPL), polyethyleneimine (PEI) and CpG oligodeoxynucleotide (“CpG ODN”). The non-toxic adjuvant cholera toxin B subunit (CTB) has been tested for adjuvancy in the investigation of mucosally targeted vaccines. When used in combination with pathogen antigens (e.g., either covalently linked to, or co-administered with the pathogen antigen), CTB can impart immunostimulatory properties characteristic of the antigen. Vaccination strategies have also been broadened to include ‘self’ proteins applied for the immunological suppression of autoimmunity. When CTB is linked to an autoantigen, the outcome might be considered paradoxical. In type 1 diabetes, self proteins become strongly immunosuppressive, while cancer CTB-autoantigen fusion proteins may exert a strong inflammatory response. (Lengridge et al., (2010) Curr. Opin. Invest. Drugs 11:919-928). However, until now, the immunostimulatory or immunosuppressive properties of the CTB subunit in vaccine protection and therapy against B. burgdorferi infection and Lyme disease have not been investigated.

In an effort to produce a reservoir-targeted vaccine, a chimeric vector construct comprising a nucleic acid sequence encoding an OspA antigen fused in-frame to a nucleic acid sequence encoding CTB as mucosal adjuvant was developed for expression in monocot plants.

Whether a recombinant Osp protein expressed by transgenic rice was capable of eliciting protective immunity was unknown and unpredictable. First, a codon-optimized rOspA amino acid sequence was altered compared to the bacterial native OspA sequence; three amino acid residues were changed to eliminate certain N-linked glycosylation sites, and it was unclear whether these changes would disrupt the three dimensional structure and destroy a major conformational protective epitope of OspA, Second, the effect of post-translational processing of rOspA in rice was not known. The simplest and most efficient way to address these questions was to administer an OspA vaccine by injection and challenge the immunized mice with needle-inoculated, culture-grown B. burgdorferi. Under this protocol, vaccine doses in the form of purified rOspA and the number of challenge organisms could be precisely controlled. Furthermore, a commercial canine vaccine could be used as positive control. If the rOspA vaccine passed these preliminary tests, efforts to formulate and optimize the vaccine for oral dosing with rOspA rice flour and challenge by tick bite would be warranted.

In the examples set forth herein, an enhanced and effective reservoir-targeted vaccine to reduce the incidence of Lyme disease based on Borrelia infection was produced by expressing a chimeric fusion protein in which the OspA gene was fused in-frame with the gene encoding CTB. The CTB.OspA fusion protein described and created herein boosted immunity in inoculated mice.

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### I. DEFINITIONS

As used in this specification, the singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

Where a range of values is provided, it is intended that each intervening value between the upper and lower limit of that range and any other stated or intervening value in that stated range is encompassed by the disclosure. For example, if a range of 1 μm to 8 μm is stated, it is intended that 2 μm, 3 μm, 4 μm, 5 μm, 6 μm, and 7 μm are also explicitly disclosed, as are the range of values greater than or equal to 1 μm and the range of values less than or equal to 8 μm.

A variety of host-expression vector systems may be utilized to express peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

“Recombinant,” when used with reference to, e.g., a cell, nucleic acid, polypeptide, expression cassette or vector, refers to a material, or a material corresponding to the natural or native form of the material, that has been modified by the introduction of a new moiety or alteration of an existing moiety, or is identical thereto but produced or derived from synthetic materials. For example, recombinant cells express genes that are not found within the native (non-recombinant) form of the cell (i.e., “exogenous nucleic acids”) or express native genes that are otherwise expressed at a different level, typically, under-expressed or not expressed at all.

Recombinant techniques can include, e.g., use of a recombinant nucleic acid such as a cDNA encoding a protein or an antisense sequence, for insertion into an expression system, such as an expression vector; the resultant construct is introduced into a cell, and the cell expresses the nucleic acid, and the protein, if appropriate. Recombinant techniques also encompass the ligation of nucleic acids to coding or promoter sequences from different sources into one expression cassette or vector for expression of a fusion protein, constitutive expression of a protein, or inducible expression of a protein.

“Exogenous” as in “exogenous nucleic acid” refers to a molecule (e.g., nucleic acid or polypeptide) that has been isolated, synthesized, and/or cloned, in a manner that is not found in nature, and/or introduced into and/or expressed in a cell or cellular environment other than or at levels or forms different than the cell or cellular environment in which said nucleic acid or protein can be found in nature. The term encompasses both nucleic acids originally obtained from a different organism or cell type than the cell type in which it is expressed, and also nucleic acids that are obtained from the same organism, cell, or cell line as the cell or organism in which it is expressed.

“Heterologous” when used with reference to a nucleic acid or polypeptide, indicates that a sequence that comprises two or more subsequences which are not found in the same relationship to each other as normally found in nature, or is recombinantly engineered so that its level of expression, or physical relationship to other nucleic acids or other molecules in a cell, or structure, is not normally found in nature. For instance, a heterologous nucleic acid is typically recombinantly produced, having two or more sequences from unrelated genes arranged in a manner not found in nature; e.g., a nucleic acid open reading frame (ORF) can be operatively linked to a promoter sequence inserted into an expression cassette, e.g., a vector. As another example, a polypeptide can be linked to tag, e.g., a detection- and purification-facilitating domain, as a fusion protein.

“Gene” refers to a nucleic acid fragment that expresses a specific protein, including regulatory sequences preceding (5′ non-coding sequences) and following (3′ non-coding sequences) the coding sequence.

“Native gene” refers to a gene as found in nature with its own regulatory sequences.

“Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature.

“Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes.

“Transgene” is a gene that has been introduced into the genome by a transformation procedure.

“Operably linked” refers to a functional relationship between two or more nucleic acid (e.g., DNA) segments. Typically, it refers to the functional relationship of a transcriptional regulatory sequence to a transcribed sequence. For example, a promoter is operably linked to a coding sequence, such as a nucleic acid, if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

“Control sequence” refers to polynucleotide sequences which are necessary to effect the expression of coding and non-coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, components whose presence can influence expression, and can also include additional components whose presence is advantageous, for example, leader sequences and fusion partner sequences.

“Modulation” refers to the capacity to either enhance or inhibit a functional property of biological activity or process (e.g., enzyme activity or receptor binding); such enhancement or inhibition may be contingent on the occurrence of a specific event, such as activation of a signal transduction pathway, and/or may be manifest only in particular cell types.

“Recombinant host cell” refers to a cell that comprises a recombinant nucleic acid molecule. Thus, for example, recombinant host cells can express genes that are not found within the native (non-recombinant) form of the cell.

“Physiological conditions” or “physiological solution” refers to an aqueous environment having an ionic strength, pH, and temperature substantially similar to conditions in an intact mammalian cell or in a tissue space or organ of a living mammal. Typically, physiological conditions comprise an aqueous solution having about 150 mM NaCl, pH 6.5-7.6, and a temperature of approximately 22-37 degrees C. Generally, physiological conditions are suitable binding conditions for intermolecular association of biological macromolecules. For example, physiological conditions of 150 mM NaCl, pH 7.4, at 37 degrees C. are generally suitable.

“Coding sequence” refers to that portion of a nucleic acid (e.g., a gene) that encodes an amino acid sequence of a protein.

“Primer” and “probe” refer to a nucleic acid molecule including DNA, RNA and analogs thereof, including protein nucleic acids (PNA), and mixtures thereof. Such molecules are typically of a length such that they are statistically unique (i.e., occur only once) in the genome of interest. Generally, for a probe or primer to be unique in the human genome, it contains at least 14, 16 or contiguous nucleotides of a sequence complementary to or identical to a gene of interest. Probes and primers can be 10, 20, 30, 50, 100 or more nucleic acids long.

“Mature protein” refers to a post-translationally processed polypeptide; i.e., one from which any pre- or propeptides present in the primary translation product has been removed. “Precursor” protein refers to the primary product of translation of mRNA; i.e., with pre- and propeptides still present. Pre- and propeptides may be but are not limited to intracellular localization signals.

“3′ non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989) Plant Cell 1:671-680.

“Translation leader sequence” refers to a nucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner and Foster (1995) Molecular Biotechnology 3:225).

“Position corresponding to” refers to a position of interest (i.e., base number or residue number) in a nucleic acid molecule or protein relative to the position in another reference nucleic acid molecule or protein. Corresponding positions can be determined by comparing and aligning sequences to maximize the number of matching nucleotides or residues, for example, such that identity between the sequences is greater than 90%, greater than 95%, greater than 96%, greater than 97%, greater than 98% or greater than 99%. The position of interest is then given the number assigned in the reference nucleic acid molecule. For example, if a particular polymorphism in Gene-X occurs at nucleotide 2073 of SEQ ID No. X, to identify the corresponding nucleotide in another allele or isolate, the sequences are aligned and then the position that lines up with 2073 is identified. Since various alleles may be of different length, the position designate 2073 may not be nucleotide 2073, but instead is at a position that “corresponds” to the position in the reference sequence.

“Transgenic” refers to any organism, prokaryotic or eukaryotic, which contains at least a cell bearing a heterologous or recombinant nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The recombinant nucleic acid molecule may be integrated within a chromosome, or it may be extrachromosomally replicating DNA.

“Associated” refers to coincidence with the development or manifestation of a disease, condition or phenotype. Association may be due to, but is not limited to, genes responsible for housekeeping functions whose alteration can provide the foundation for a variety of diseases and conditions, those that are part of a pathway that is involved in a specific disease, condition or phenotype and those that indirectly contribute to the manifestation of a disease, condition or phenotype.

General and specific techniques for producing proteins from plant cells may be obtained from the following applications, each of which is incorporated herein in its entirety by reference: U.S. patent application Ser. No. 09/847,232 (“Plant Transcription Factors and Enhanced Gene Expression”); U.S. patent application Ser. No. 10/077,381 (“Expression of Human Milk Proteins in Transgenic Plants”); U.S. patent application Ser. No. 10/411,395 (“Human Blood Proteins Expressed in Monocot Seeds”); U.S. patent application Ser. No. 10/639,779 (“Production of Human Growth Factors in Monocot Seeds”); U.S. patent application Ser. No. 10/639,781 (“Method of Making an Anti-infective Composition for Treating Oral Infections”); and international application no. PCT/US2004/041083 (“High-level Expression of Fusion Polypeptides in Plant Seeds Utilizing Seed-Storage Proteins as Fusion Carriers”).

A “plant cell” refers to any cell derived from a plant, including undifferentiated tissue (e.g., callus) as well as plant seeds, pollen, propagules, embryos, suspension cultures, meristematic regions, leaves, roots, shoots, gametophytes, sporophytes and microspores.

The plant can be a monocot plant. The plant is often a cereal, selected from the group consisting of rice, barley, wheat, oat, rye, corn, millet, triticale and sorghum. The term “mature plant” refers to a fully differentiated plant.

Plant cells or tissues are transformed with expression constructs using a variety of standard techniques. In some embodiments, the vector sequences are stably integrated into the host genome. Suitable plants are those that have been transformed with a CTB.OspA expression vector, or have been grown from a plant cell that has been transformed with a CTB.OspA expression vector, in accordance with the methods described herein, and express a CTB.OspA fusion protein as a result of the transformation. Also suitable are plants that have been transformed with a CTB.OspA expression vector, or have been grown from a plant cell that has been transformed with a CTB.OspA expression vector, that are fertile and phenotypically normal and express a CTB.OspA fusion protein.

As used herein, the terms “transformed” or “transgenic” with reference to a host cell means the host cell contains a non-native or heterologous or introduced nucleic acid sequence that is absent from the native host cell. Further, “stably transformed” in the context of the present disclosure means that the introduced nucleic acid sequence is maintained through two or more generations of the host, which may be due to integration of the introduced sequence into the host genome.

According to another aspect of the disclosure, plants that have been transformed with the CTB.OspA expression vector exhibit growth that is comparable to a wild-type plant of the same species, or exhibit fertility that is comparable to a wild-type plant of the same species, or both. A transformed plant that exhibits comparable growth to a wild-type plant may produce at least 80% of the amount of total biomass produced by a wild-type plant grown under similar conditions, such as location (e.g., greenhouse, field, etc.), soil type, nutrients, water, and exposure to sunlight. The transformed plant may produce at least 85%, or at least 90%, or at least 95% of the amount of total biomass produced by a wild-type plant grown under similar conditions. A transformed plant that exhibits comparable fertility to a wild-type plant may produce at least 80% of the amount of offspring produced by a wild-type plant grown under similar conditions, such as location (e.g., greenhouse, field, etc.), soil type, nutrients, water, and exposure to sunlight. In some embodiments, the transformed plant produces at least 85%, at least 90%, or at least 95% of the amount of offspring produced by a wild-type plant grown under similar conditions.

According to a further aspect of the disclosure, the plants transformed with the CTB.OspA gene construct are comparable to a wild-type plant of the same species and express the CTB.OspA protein as a result of the transformation. In some embodiments, the transformed plants express the CTB.OspA fusion protein at high levels, e.g., 2%, 3%, 5%, 8%, 9%, 10%, or 20% or greater of the total soluble protein in the seeds of the plant.

The method used for transformation of host plant cells is not critical to the present disclosure. For commercialization of the heterologous peptide or polypeptide expressed in accordance with the present disclosure, the transformation of the plant can be permanent, L e., by integration of the introduced expression constructs into the host plant genome, so that the introduced constructs are passed onto successive plant generations. The skilled artisan will recognize that a wide variety of transformation techniques exist in the art, and new techniques are continually becoming available.

Any technique that is suitable for the target host plant may be employed within the scope of the present disclosure. For example, the constructs can be introduced in a variety of forms including, but not limited to, as a strand of DNA, in a plasmid, or in an artificial chromosome. The introduction of the constructs into the target plant cells can be accomplished by a variety of techniques, including, but not limited to calcium-phosphate-DNA co-precipitation, electroporation, microinjection, Agrobacterium-mediated transformation, liposome-mediated transformation, protoplast fusion or microprojectile bombardment. The skilled artisan can refer to the literature for details and select suitable techniques for use in the methods of the present disclosure.

Transformed plant cells are screened for the ability to be cultured in selective media having a threshold concentration of a selective agent. Plant cells that grow on or in the selective media are typically transferred to a fresh supply of the same media and cultured again. The explants are then cultured under regeneration conditions to produce regenerated plant shoots. After shoots form, the shoots can be transferred to a selective rooting medium to provide a complete plantlet. The plantlet may then be grown to provide seed, cuttings, or the like for propagating the transformed plants. Suitable selectable markers for selection in plant cells include, but are not limited to, antibiotic resistance genes, such as kanamycin (nptll), G418, bleomycin, hygromycin, chloramphenicol, ampicillin, tetracycline, and the like. Additional selectable markers include a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulphonylurea resistance. The particular marker gene employed is one which allows for selection of transformed cells as compared to cells lacking the nucleic acid which has been introduced. In some embodiments, the selectable marker gene is one that facilitates selection at the tissue culture stage, e.g., an nptll, hygromycin or ampicillin resistance gene. Thus, the particular marker employed is not essential in the present compositions and methods.

The fusion protein may also be engineered to comprise at least one selective purification tag and/or at least one specific protease cleavage site for eventual release of the OspA protein from the seed storage protein fusion partner, fused in translation frame between the OspA protein and the seed storage protein. In some embodiments, the specific protease cleavage site may comprise enterokinase (ek), Factor Xa, thrombin, V8 protease, Genenase™, α-lytic protease or tobacco etch virus (TEV) protease. The fusion protein may also be cleaved chemically.

The expression of the heterologous peptide or polypeptide may be confirmed using standard analytical techniques such as Western blot, ELISA, PCR, HPLC, NMR, or mass spectroscopy, together with assays for a biological activity specific to the particular protein being expressed.

By “host cell” is meant a cell containing a vector and supporting the replication and/or transcription and/or expression of a vector-encoded nucleic acid sequence. According to the present disclosure, the host cell is a plant cell. Other host cells may be used as secondary hosts, including bacterial, yeast, insect, amphibian or mammalian cells, to move DNA to a desired plant host cell.

The term “seed” refers to all seed components, including, for example, the coleoptile and leaves, radicle and coleorhiza, scutulum, starchy endosperm, aleurone layer, pericarp and/or testa, either during seed maturation and seed germination. In the context of the present disclosure, the term “seed” and “grain” is used interchangeably.

“Seed components” refers to carbohydrate, protein, and lipid components extractable from seeds, typically mature seeds.

The term “seed product” includes, but is not limited to, seed fractions such as de-hulled whole seed, a flour (seed that has been de-hulled by milling and ground into a powder), a seed extract, a protein extract (where the protein fraction of the flour has been separated from the carbohydrate fraction), a malt (including malt extract or malt syrup) and/or a purified protein fraction derived from the transgenic grain.

“Seed maturation” refers to the period starting with fertilization in which metabolizable reserves, e.g., sugars, oligosaccharides, starch, phenolics, amino acids, and proteins, are deposited, with and without vacuole targeting, to various tissues in the seed (grain), e.g., endosperm, testa, aleurone layer, and scutellar epithelium, leading to grain enlargement, grain filling, and ending with grain desiccation.

“Plant-derived” refers to a recombinant expression product (nucleic acid or polypeptide) that is not endogenous to the plant, but is expressed in the transgenic plant upon introduction of a recombinant nucleic acid sequence.

The seed storage protein can be from a monocot plant. In some embodiments, the seed storage protein is selected from the group consisting of rice globulins, rice glutelins, oryzins, prolamines, barley hordeins, wheat gliadins and glutenins, maize zeins and glutelins, oat glutelins, sorghum kafirins, millet pennisetins, or rye secalins. For example, rice globulin and rice glutelin are suitable. The seed storage protein may be at the N-terminal or C-terminal side of the OspA protein in the fusion protein. In some embodiments, the seed storage protein is located at the N-terminal side of the OspA protein.

“Maturation-specific protein promoter” refers to a promoter exhibiting substantially upregulated activity (greater than 25%) during seed maturation. The promoter may be from a maturation-specific monocot plant storage protein or an aleurone- or embryo-specific monocot plant gene. Other promoters may be used, however, and the choice of a suitable promoter is within the skill of those in the art. As such, the promoter can be a member selected from the group consisting of rice globulins, glutelins, oryzins and prolamines, barley hordeins, wheat gliadins and glutenins, maize zeins and glutelins, oat glutelins, sorghum kafirins, millet pennisetins, rye secalins, lipid transfer protein Ltp1, chitinase Chi26 and Em protein Emp1. In some embodiments, the promoter is selected from the group consisting of rice globulin Glb promoter and rice glutelin Gt1 promoter.

The seed-specific signal sequence used to replace the signal peptide from OspA may be from a monocot plant, although other signal sequences may be utilized. In some embodiments, the monocot plant seed-specific signal sequence is associated with a gene selected from the group consisting of glutelins, prolamines, hordeins, gliadins, glutenins, zeins, albumin, globulin, ADP glucose pyrophosphorylase, starch synthase, branching enzyme, Em, and lea. In some embodiments, the monocot plant seed-specific signal sequence is a rice glutelin Gt1 signal sequence. Other monocot plant seed-specific signal sequence are associated with genes selected from the group consisting of α-amylase, protease, carboxypeptidase, endoprotease, ribonuclease, DNase/RNase, (1-3)-β-glucanase, (1-3)(1-4)-β-glucanase, esterase, acid phosphatase, pentosamine, endoxylanase, β-xylopyranosidase, arabinofuranosidase, β-glucosidase, (1-6)-β-glucanase, perioxidase, and lysophospholipase.

The promoter and signal sequence may be selected from those discussed supra. The type of promoter and signal sequence is not critical to this disclosure. In some embodiments, the signal sequence targets the attached fusion protein to a location such as an intracellular compartment, such as an intracellular vacuole or other protein storage body, mitochondria, or endoplasmic reticulum, or extracellular space, following secretion from the host cell.

The term “biological activity” refers to any biological activity typically attributed to a nucleic acid or protein by those skilled in the art. Examples of biological activities are enzymatic activity, ability to dimerize, fold or bind another protein or nucleic acid molecule, etc.

The nucleic acids of the present disclosure may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand.

As used herein, the Borrelia spp. may be selected from the group consisting of B. burgdorferi sensu stricto S-1-10 and C-1-11, Borrelia afzelii BV1, Borrelia garinii LV4, B. afzelii PKo, B. valaisiana strains, B. burgdorferi sensu lato LV5, B. burgdorferi PKo, B. burgdorferi PBi, B. burgdorferi B31, B. burgdorferi ZS7, and B. burgdorferi N40.

“Heterologous nucleic acid” refers to nucleic acid which has been introduced into plant cells from another source, or which is from a plant source, including the same plant source, but which is under the control of a promoter that does not normally regulate expression of the heterologous nucleic acid. “Heterologous peptide or polypeptide” is a peptide or polypeptide encoded by a heterologous nucleic acid. The peptides or polypeptides include OspA proteins, such as B. burgdorferi OspA proteins. OspA proteins include, but are not limited to, those derived from B. burgdorferi sensu stricto S-1-10 and C-1-11, Borrelia afzelii BV1, Borrelia garinii LV4, B. afzelii PKo, B. valaisiana strains, B. burgdorferi sensu lato LV5, B. burgdorferi PKo, B. burgdorferi PBi, B. burgdorferi B31, B. burgdorferi ZS7, and B. burgdorferi N40. Any B. burgdorferi OspA proteins, including those yet to be identified, may be used in accordance with the compositions and methods of the present disclosure.

“Percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

While all of the above mentioned algorithms and programs are suitable for a determination of sequence alignment and % sequence identity, for purposes of the disclosure herein, determination of % sequence identity will typically be performed using the BESTFIT or GAP programs in the GCG Wisconsin Software package (Accelrys, Madison Wis.), using default parameters provided.

The open reading frame of the B. burgdorferi OspA gene consists of 822 nucleotides corresponding to a protein of 273 amino acids, including 16 amino acids as a signal peptide, and the protein has a calculated molecular mass of 29.6 kDa. High level expression of this protein in tobacco cells is lethal to the plant (see, e.g., FEBS J 274(21):5749-58 (2007)). The proteins contain a variable middle region, whereas the N and the C terminus are conserved. There is an unexpectedly high level of dissimilarity between the various OspA genes, and this may make it important to incorporate more than one Osp protein into a vaccine in order to confer optimum immunity.

As will be understood by those of skill in the art, in some cases it may be advantageous to use a nucleotide sequences possessing non-naturally occurring codons. Codons preferred by a particular eukaryotic host can be selected, for example, to increase the rate of expression or to produce recombinant RNA transcripts having desirable properties, such as a longer half-life, than transcripts produced from naturally occurring sequence. As an example, it has been shown that codons for genes expressed in rice are rich in guanine (G) or cytosine (C) in the third codon position (Huang et al., (1990) J. CAASS 1: 73-86). Changing low G+C content to a high G+C content has been found to increase the expression levels of foreign protein genes in barley grains (Horvath et al., (2000) Proc. Natl. Acad. Sci. USA 97: 1914-19). If a rice plant is selected, the genes employed in the present disclosure may be based on the rice gene codon bias (Huang et al., supra) along with the appropriate restriction sites for gene cloning. These codon-optimized genes may be linked to regulatory and secretion sequences for seed-directed expression and these chimeric genes then inserted into the appropriate plant transformation vectors.

Because the recombinant Osp protein(s) of the present disclosure are produced in plants, they may include plant glycosyl groups at one or more of the available N-glycosylation sites of the Osp protein(s). For example, in one embodiment of the disclosure, a glycosylated CTB.OspA protein(s) is produced in monocot seeds, such as rice, barley, wheat, oat, rye, corn, millet, triticale and sorghum. Most OspA proteins include five sites for glycosylation. When produced by the methods of the disclosure, the CTB.OspA fusion protein may be glycosylated at all five sites, at any four sites, at any three sites, at any two sites, or at any single glycosylation site. If a variant of an Osp protein having a different number of N-glycosylation sites is utilized, it may be glycosylated at all or less than all of the N-glycosylation sites. Optionally, any or all plant glycosyl groups may be removed.

“Position corresponding to” refers to a position of interest (i.e., base number or residue number) in a nucleic acid molecule or protein (or polypeptide or peptide fragment) relative to the position in another reference nucleic acid molecule or protein. Corresponding positions can be determined by comparing and aligning sequences to maximize the number of matching nucleotides or residues, for example, such that identity between the sequences is greater than 90%, greater than 95%, greater than 96%, greater than 97%, greater than 98% or greater than 99%. The position of interest is then given the number assigned in the reference nucleic acid molecule. For example, it is shown herein that a particular polymorphism in Gene-Y occurs at nucleotide 2073 of SEQ ID No. X. To identify the corresponding nucleotide in another allele or isolate, the sequences are aligned and then the position that lines up with 2073 is identified. Since various alleles may be of different length, the position designate 2073 may not be nucleotide 2073, but instead is at a position that “corresponds” to the position in the reference sequence.

As used herein, a “variant” is a nucleic acid, protein or peptide which is not identical to, but has significant homology (for example, 80%, 85%, 90%, 91%, 92%, 93%, 94%, 95%, 96%, 97%, 98% or 99% sequence identity) over the entire length of the wild type nucleic acid or amino acid sequence, as exemplified by sequences in the public sequence databases, such as GenBank. As used herein, a “protein, polypeptide or peptide fragment thereof” means the full-length protein or a portion of it having a wild type amino acid sequence usually at least 6, 7, 8, 9, 10, 11, 12, 13, 14 or 15 amino acids in length.

As used herein, a “mutant” is a mutated protein designed or engineered to alter properties or functions relating to glycosylation, protein stabilization and/or ligand binding.

As used herein, the terms “native” or “wild-type” relative to a given cell, polypeptide, nucleic acid, trait or phenotype, refers to the form in which that is typically found in nature.

The disclosure provides CTB.OspA protein recombinantly produced in a host plant seed. As used herein, “high levels of protein expression” means that the plant-expressed CTB.OspA protein comprises about 2% or greater of the total soluble protein in the seed. Thus, for example, the yield of total soluble protein which comprises the CTB.OspA protein targeted for production can be about 3% or greater, about 5% or greater, about 8% or greater, about 9% or greater, about 10% or greater, or about 20% or greater, of the total soluble protein found in the recombinantly engineered plant seed. Alternatively, the phrase “high yield expression” can mean that the level of expression of the recombinant Osp protein in transgenic plant cells, plants or mature seeds is sufficiently high that a flour, extract or malt can be prepared from the seed directly without the need to purify the expressed protein.

The CTB.OspA protein constitutes at least 0.01 weight percent in the harvested seeds. In some embodiments, the CTB.OspA protein constitutes at least 0.05 weight percent, and in some embodiments, at least 0.1 weight percent in the harvested seeds. Generally, “total soluble proteins” refers to the total amount of protein in a solution used to extract protein from a tissue. The phrase “total storage proteins” can encompass extractable and non-extractable protein. An average rice grain seed weight is 20-30 mg.

Suitable expression vectors for the production of CTB.OspA or variant or fragment thereof are vectors which are capable of replicating in a host organism upon transformation. The vector may either be one which is capable of autonomous replication, such as a plasmid, or one which is replicated with the host chromosome, such as a bacteriophage. Examples of suitable vectors which have been widely employed are pBR322 and related vectors as well as pUC vectors and the like. Examples of suitable bacteriophages include M13 and lambda phage.

The organism harboring the vector carrying the DNA fragment or part thereof may be any organism which is capable of expressing said DNA fragment. The organism can be a microorganism such as a bacterium. Gram-positive as well as gram-negative bacteria may be employed. Especially a gram-negative bacterium such as E. coli is useful, but also gram-positive bacteria such as B. subtilis and other types of microorganisms such as yeasts or fungi or other organisms conventionally used to produce recombinant DNA products may be used. Another type of organism which may be used to express CTB.OspA or a part thereof is a higher eukaryotic organism or cell, including a plant and mammal cell. However, also higher organisms such as animals, e.g. sheep, cattle, goats, pigs, horses and domestic animals, including cats and dogs, are contemplated to be useful as host organisms for the production of CTB.OspA or a part thereof.

When a higher organism, e.g. an animal, is employed for the production of CTB.OspA or a part thereof, conventional transgenic techniques may be employed. These techniques comprise inserting the DNA fragment or one or more parts thereof into the genome of the animal in such a position that CTB.OspA or part thereof is expressed together with a polypeptide which is inherently expressed by the animal, in many cases, a polypeptide which is easily recovered from the animal, e.g. a polypeptide which is secreted by the animal, such as a milk protein or the like. Alternatively, the DNA fragment could be inserted into the genome of the animal in a position allowing the gene product of the expressed DNA sequence to be retained in the animal body so that a substantial steady immunization of the animal takes place. When a microorganism is used for expressing the DNA fragment, the cultivation conditions will typically depend on the type of microorganism employed, and the skilled art worker will know which cultivation method to choose and how to optimize this method.

The production of Osp proteins or a part thereof by recombinant techniques has a number of advantages: it is possible to produce OspA or CTB.OspA fusion protein or a polypeptide part thereof by culturing non-pathogenic organisms or other organisms which do not affect the immunological properties of the OspA or CTB.OspA fusion protein or a polypeptide part thereof, it is possible to produce the protein in higher quantities than those obtained when recovering Osp proteins from any wild type fractions, and it is possible to produce parts of Osp proteins which may not be isolated from B. burgdorferi strains. The higher quantities of OspA or CTB.OspA fusion protein or a polypeptide part thereof may for instance be obtained by using high copy number vectors for cloning the DNA fragment or by using a strong promoter to induce a higher level of expression than the expression level obtained with the promoters P1 and P2 present on the DNA fragment disclosed herein. By use of recombinant DNA techniques for producing the Osp protein, OspA or CTB.OspA fusion protein or a polypeptide part thereof, unlimited amounts of a substantially pure protein or polypeptide which is not “contaminated” with other components which are normally present in B. burgdorferi isolates may be obtained. Thus, it is possible to obtain a substantially pure Osp protein, i.e. OspA or CTB.OspA fusion protein or a polypeptide part thereof which is not admixed with other B. burgdorferi proteins which have an adverse effect when present in a vaccine or a diagnostic agent in which the OspA is an intended constituent. A substantially pure OspA or CTB.OspA fusion protein or a polypeptide part thereof has the additional advantage that the exact concentration thereof in a given vaccine preparation is known so that an exact dosage may be administered to the individual to be immunized. An important aspect of the present disclosure concerns a vaccine for the immunization of an animal, such as a mammal, including a human being, against Lyme disease, which vaccine comprises an immunologically effective amount of any one of the above defined fractions or combinations thereof together with an immunologically acceptable carrier or vehicle. It should be understood that the term “animal” includes the human animal.

As used herein, the term “purifying” is used interchangeably with the term “isolating” and generally refers to any separation of a particular component from other components of the environment in which it is found or produced. For example, purifying a recombinant protein from plant cells in which it was produced typically means subjecting transgenic protein-containing plant material to separation techniques such as sedimentation, centrifugation, filtration, and chromatography. The results of any such purifying or isolating step(s) may still contain other components as long as the results have less of the other components (“contaminating components”) than before such purifying or isolating step(s).

The compounds of the present disclosure can be purified or “at least partially purified” by art-known techniques such as reverse phase chromatography high performance liquid chromatography, ion exchange chromatography, gel electrophoresis, affinity chromatography and the like. The actual conditions used to purify a particular compound will depend, in part, on synthesis strategy and on factors such as net charge, hydrophobicity, hydrophilicity, etc., and will be apparent to those having skill in the art.

As used herein, the terms “reservoir” or “reservoir species” or “reservoir animal(s)” with reference to Lyme disease or B. burgdorferi means a non-human population that serves as a host for Lyme disease causing agents, particularly B. burgdorferi.

As used herein, the term “vector” with reference to the transmission of Lyme disease and the disease cycle refers to agents such as ticks that commonly transmit a Lyme disease causing agent from one host to another.

As used herein, the term “disease cycle” with reference to Borellia spp. infection and refers to the process by which a vector, such as a tick, transmits a Lyme disease-causing agent such as an Osp protein to a suitable host, such as a rodent. The cycle can be a complete life cycle or any portion of that cycle. The life cycle of B. burgdorferi is complex, and may require ticks, rodents, and deer at various points. For example a complete cycle involves Borellia infection of a rodent host by a tick, which tick feeds on and transfers the Lyme disease-causing agents to other vectors such as deer or humans by biting them. Mice are the primary reservoir for the bacteria; Ixodes ticks then transmit the B. burgdorferi infection to deer. Hard ticks have a variety of life histories with respect to optimizing their chance of contact with an appropriate host to ensure survival. The life stages of soft ticks are not readily distinguishable. The first life stage to hatch from the egg, a six-legged larva, takes a blood meal from a host, and molts to the first nymphal stage. Unlike hard ticks, many soft ticks go through multiple nymphal stages, gradually increasing in size until the final molt to the adult stage. The life cycle of the deer tick comprises three growth stages: the larva, nymph and adult. The life-cycle concept encompassing reservoirs and infections in multiple hosts has recently been expanded to encompass forms of the spirochete which differ from the motile corkscrew form, and these include cystic spheroplast-like forms, straight non-coiled bacillary forms which are immotile due to flagellin mutations and granular forms, coccoid in profile. The model of Plasmodium species malaria, with multiple parasitic profiles demonstrable in various host insects and mammals, is a hypothesized model for a similarly complex proposed Borrelia spirochete life cycle. Whereas B. burgdorferi is most associated with deer tick and the white footed mouse, B. afzelli is most frequently detected in rodent-feeding vector ticks, and B. garinii and B. valaisiana appear to be associated with birds. Both rodents and birds are competent reservoir hosts for Borrelia burgdorferi sensu stricto. The resistance of a genospecies of Lyme disease spirochetes to the bacteriolytic activities of the alternative immune complement system of various host species may determine its reservoir host association.

The term “prevention,” “amelioration” or “treatment” of infection by a Borrelia species refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation.

The active compound(s) described herein, or compositions thereof, will generally be used in an amount effective to treat or prevent the particular disease being treated. The compound(s) may be administered therapeutically to achieve therapeutic benefit or prophylactically to achieve prophylactic benefit. By therapeutic benefit is meant eradication or amelioration of the underlying disorder being treated, e.g., eradication or amelioration of the underlying allergy, atopic dermatitis, atopic eczema or atopic asthma, and/or eradication or amelioration of one or more of the symptoms associated with the underlying disorder such that the patient reports an improvement in feeling or condition, notwithstanding that the patient may still be afflicted with the underlying disorder. For example, administration of an active compound to a patient suffering from an allergy provides therapeutic benefit not only when the underlying allergic response is eradicated or ameliorated, but also when the patient reports a decrease in the severity or duration of the symptoms associated with the allergy following exposure to the allergen. Therapeutic benefit also includes halting or slowing the progression of the disease, regardless of whether improvement is realized.

For prophylactic administration, the active compound may be administered to a patient at risk of developing a disorder characterized by, caused by or associated with IgE production and/or accumulation, such as the various disorders previously described. For example, if it is unknown whether a patient is allergic to a particular drug, the active compound may be administered prior to administration of the drug to avoid or ameliorate an allergic response to the drug. Alternatively, prophylactic administration may be applied to avoid the onset of symptoms in a patient diagnosed with the underlying disorder. For example, an active compound may be administered to an allergy sufferer prior to expected exposure to the allergen. Active compounds may also be administered prophylactically to healthy individuals who are repeatedly exposed to agents known to induce an IgE-related malady to prevent the onset of the disorder. For example, an active compound may be administered to a healthy individual who is repeatedly exposed to an allergen known to induce allergies, such as latex allergy, in an effort to prevent the individual from developing an allergy.

The amount of active compound(s) administered will depend upon a variety of factors, including, for example, the particular indication being treated, the mode of administration, whether the desired benefit is prophylactic or therapeutic, the severity of the indication being treated and the age and weight of the subject animal/patient, the bioavailability of the particular active compound, etc. Determination of an effective dosage is well within the capabilities of those skilled in the art. Initial dosages may be estimated initially from in vitro assays.

“Breaking a Lyme disease cycle” according to the present disclosure means controlling pathogen prevalence in one or more reservoir animals, thereby interrupting the normal life cycle and reducing the rate of host infection.

The one or more OspA proteins can be further formulated together with one or more pharmaceutically acceptable excipients to produce a pharmaceutical composition. The term “excipient” or “vehicle” as used herein means any substance, not itself a therapeutic agent, used as a carrier for delivery of a therapeutic agent and suitable for administration to a subject, e.g. a mammal or added to a pharmaceutical composition to improve its handling or storage properties or to permit or facilitate formation of a dose unit of the composition into a discrete article such as a capsule or tablet suitable for oral administration. Excipients and vehicles include any such materials known in the art, e.g., any liquid, gel, solvent, liquid diluent, solubilizer, or the like, which is nontoxic and which does not interact with other components of the composition in a deleterious manner. The excipients may include standard pharmaceutical excipients, and may also include any components that may be used to prepare foods and beverages for human and/or animal consumption, or bait formulations.

For example, excipients include, by way of illustration and not limitation, diluents, disintegrants, binding agents, adhesives, wetting agents, lubricants, glidants, crystallization inhibitors, surface modifying agents, substances added to mask or counteract a disagreeable taste or odor, flavors, dyes, fragrances, and substances added to improve appearance of the composition. Excipients employed in compositions of the disclosure can be solids, semi-solids, liquids or combinations thereof. Compositions of the disclosure containing excipients can be prepared by any known technique of pharmacy that comprises admixing an excipient with a drug or therapeutic agent. Other excipients such as colorants, flavors, and sweeteners, which may make the oral formulations of the present disclosure more desirable to animal hosts of B. burgdorferi tick vectors can also be used in compositions of the present disclosure.

“Permeant,” “drug,” or “pharmacologically active agent” or any other similar term means any chemical or biological material or compound, inclusive of peptides, suitable for transmucosal administration by the methods previously known in the art and/or by the methods taught in the present disclosure, that induces a desired biological or pharmacological effect, which may include but is not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating the disease from the organism. The effect may be local, such as providing for a local anaesthetic effect, or it may be systemic. This disclosure is not drawn to novel permeants or to new classes of active agents. Rather it is limited to the mode of delivery of agents or permeants which exist in the state of the art or which may later be established as active agents and which are suitable for delivery by the present disclosure. Such substances include broad classes of compounds normally delivered into the body, including through body surfaces and membranes, including skin. In general, this includes but is not limited to: antiinfectives such as antibiotics and antiviral agents; analgesics and analgesic combinations; anorexics; antihelminthics; antiarthritics; antiasthmatic agents; anticonvulsants; antidepressants; Antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations including potassium and calcium channel blockers, beta-blockers, alpha-blockers, and antiarrhythmics; antihypertensives; diuretics and antidiuretics; vasodilators including general coronary, peripheral and cerebral; central nervous system stimulants; vasoconstrictors; cough and cold preparations, including decongestants; hormones such as estradiol and other steroids, including corticosteroids; hypnotics; immunosuppressives; muscle relaxants; parasympatholytics; psychostimulants; sedatives; and tranquilizers. By the method of the present disclosure, both ionized and nonionized drugs may be delivered, as can drugs of either high or low molecular weight.

“Buccal” drug delivery is meant delivery of a drug by passage of a drug through the buccal mucosa into the bloodstream. Buccal drug delivery may be effected herein by placing the buccal dosage unit on the upper gum or opposing inner lip area of the individual undergoing drug therapy.

The oral formulations according to the present disclosure can be prepared in any manner suitable to deliver the Osp protein(s) in order to induce an immune response in the organism to which the formulation is administered. Conventional blending, tableting, and encapsulation techniques known in the art can be employed. Oral dosage forms are suitable for administering the one or more Osp protein(s) produced in accordance with the present disclosure due to their ease of administration; however, parenteral formulations containing the recombinant Osp protein(s) of the present disclosure are also envisioned and these may be prepared in accordance with known methods. Examples of dosage forms for administration to a human include a tablet, a caplet, a hard or soft capsule, a lozenge, a cachet, a dispensable powder, granules, a suspension or solution, an elixir, a liquid, or any other form reasonably adapted for oral administration. Examples of dosage forms for administration to an animal include foods, liquids, baits, and any other compositions that are likely to be consumed by the animal to be vaccinated.

According to one aspect of the disclosure, oral vaccine formulations including more than one type of Osp protein may be provided. This approach is believed to be beneficial in conferring immunity against Lyme disease-causing agents, particularly B. burgdorferi spp., because it may induce the production of a variety of different antibodies. The oral formulations for vaccinating against Lyme disease may include recombinant OspA proteins derived from one or more of B. burgdorferi sensu stricto S-1-10 and C-1-11, Borrelia afzelii BV1, Borrelia garinii LV4, B. afzelii PKo, B. valaisiana strains, B. burgdorferi sensu law LV5, B. burgdorferi PKo, B. burgdorferi PBi, B. burgdorferi B31, B. burgdorferi ZS7, and B. burgdorferi N40, but they are not limited to these strains. Any B. burgdorferi OspA proteins, including those yet to be identified, may be used in the oral vaccine formulations of the present disclosure.

When oral formulations are prepared from a genetically-modified monocot seed, it is possible to first purify the recombinant Osp protein(s), and then incorporate them into a food, beverage, or bait formulation. In accordance with this aspect of the disclosure, any components that are added to the genetically-modified monocot seed to form a food, beverage, or bait formulation may be considered excipients. One of the benefits of the present disclosure is the ability to directly utilize the genetically-modified monocot seed in the production of such a food, beverage, or bait formulations without first purifying the Osp protein(s). This is possible at least in part because of the relatively high levels of the recombinant Osp protein(s) in the seeds produced by the methods of the present disclosure.

The oral formulations containing Osp protein(s) according to the present disclosure may be administered in any dose adequate to vaccinate an animal, i.e., induce an immune response in said animal to Osp protein(s), thereby protecting the animal from infection by Lyme disease-causing agents, particularly B. burgdorferi spp. This in turn prevents the spread of Lyme disease to other animals or humans, by preventing or eliminating the presence of Lyme disease causing agents from vectors that feed upon the infected animal, particularly ticks. In one embodiment of the present disclosure, the oral formulation is administered in doses of from about 0.1 microgram (μg)/day to about 100 mg/day, about 1 μg/day to about 10 mg/day, about 5 μg/day to about 5 mg/day, about 10 μg/day to about 1 mg/day, or about 25 μg/day to about 0.5 mg/day.

According to some embodiments, it is also possible to prepare parenterally-administered vaccines for Lyme disease using the recombinant Osp protein(s) produced in monocot seeds by first purifying the Osp protein(s) from the seeds, and then incorporating them into a standard parenteral vaccine formulation using techniques known in the art. Such parenteral vaccines may be administered in any amount sufficient to confer immunity to Lyme disease-causing agents, particularly B. burgdorferi spp.

For example, to help the release of Osp protein(s) in small intestine, the oral formulations may be tableted or pelleted, or encapsulated, and may be enteric-coated. Enteric coating prevents a tablet or capsule from dissolving before it reaches the small intestine. Alternatively the material may be spheronized into microparticles and may be enterically coated. Spheroids may be produced in the size range of 250 μm to 850 μm. Enteric coatings are known to be selectively insoluble substances that do not dissolve in the acidic environment of the stomach, but dissolve in the higher pH of the small intestine, resulting in a specific release of OspA protein(s) in the small intestine.

The active compound(s) described herein will provide therapeutic or prophylactic benefit without causing substantial toxicity. Toxicity of the active compound(s) may be determined using standard pharmaceutical procedures. The dose ratio between toxic and therapeutic (or prophylactic) effect is the therapeutic index. Active compound(s) that exhibit high therapeutic indices are suitable.

The term “immunization” is understood to comprise the process of evoking a specific immunologic response with the expectation that this will result in humoral, and/or secretory, and/or cell-mediated immunity to infection with Borrelia species, i.e. immunity is to be understood to comprise the ability of the individual to resist or overcome infection or to overcome infection more easily when compared to individuals not being immunized or to tolerate the infection without being clinically affected. Thus, the immunization according to the present disclosure is a process of increasing resistance to infection with Borrelia species.

In another aspect, the present disclosure relates to a vaccine comprising an immunogenically effective amount of a polypeptide as described above, i.e. the entire OspA or CTB.OspA fusion protein or a polypeptide portion or an immunogenic part thereof, e.g. an epitope or an antigenic determinant of the OspA protein. Also, a vaccine comprising an immunogenically effective amount of one or more of the proteins present in any of the purified fractions may be of interest. Antibodies against the polypeptides with a molecular weight of 55 and 85 kd have been found in sera from patients infected with B. burgdorferi strains, indicating that these proteins exert an immunological activity. The molecular weights of the proteins given above are the molecular weights of the proteins isolated from the B. burgdorferi strain B31 (ATCC 35210), and proteins isolated from other B. burgdorferi strains corresponding to these proteins, although not having the same molecular weights, are of course also interesting as vaccine components. A vaccine comprising one or more of the polypeptides described above, i.e. OspA or CTB.OspA fusion protein or a polypeptide part thereof, in combination with one or more of the other Osp proteins also may be useful. Also, vaccines constituting one or more of the polypeptides described above and immunologically active components from other organisms may be desirable.

The immunologically acceptable carrier or vehicle being part of the vaccine may be any carrier or vehicle usually employed in the preparation of vaccines. Thus, the vehicle may be a diluent, a suspending agent or other similar agents. The vaccine may be prepared by mixing an immunogenically effective amount of any of the purification fractions, the polypeptides defined above, one or more proteins of the fractions or a combination of any of these with the vehicle in an amount resulting in the desired concentration of the immunogenically effective component of the vaccine. The amount of immunogenically effective component in the vaccine will of course depend on the animal to be immunized, e.g. the age and the weight of the animal, as well as the immunogenicity of the immunogenic component present in the vaccine. For most purposes, an amount of the immunogenic component of the vaccine will be in the range of 5-500 μg. The methods of preparation of vaccines according to the present disclosure are designed to ensure that the identity and immunological effectiveness of the specific molecules are maintained and that no unwanted microbial contaminants are introduced. The final products are distributed under aseptic conditions into suitably sterile containers which are then sealed to exclude extraneous microorganisms.

As stated above, the OspA or CTB.OspA fusion protein or a polypeptide part thereof may be prepared by recombinant DNA techniques or by solid or liquid phase peptide synthesis. Polypeptides prepared in this manner are especially desirable as vaccine components as these polypeptides are essentially free from other contaminating components which will influence the immunogenic properties of the polypeptides. Thus, polypeptides prepared by recombinant DNA techniques or by solid or liquid phase peptide synthesis may be obtained in a substantially pure form which is very desirable for vaccine purposes. When proteins or other immunogenically active components present in any of the purification fractions are employed as vaccine constituents, these may advantageously be recovered from the fractions by any conventional method, e.g. a method in which antibodies, such as monoclonal antibodies, reactive with the proteins or other immunologically active components of fractions are immobilized to a matrix, the matrix is contacted with the fraction in question, washed, and finally the antigen-antibody complex fixed to the matrix is treated so as to release the B. burgdorferi related proteins or other immunologically active components in a purified form. The B. burgdorferi related proteins may also be isolated by means of column affinity chromatography involving antibodies fixed to the column matrix.

The phrase “converting a non-mucosally-active microbial antigen to a mucosally-active vaccine” means that the recombinant microbial antigen (1) is produced in plant cells; (2) is immune-active and is capable of stimulating protective antibody production for protection of a subject from infection upon parenteral administration (e.g., subcutaneous injection); (3) is not immunostimulatory when provided via a mucosal route of administration (e.g., orally), whether the antigen is administered alone or mixed with (but not fused to) a mucosal adjuvant; and (4) becomes mucosally-active and immunostimulatory, stimulating protective antibody production and protecting animals from infection. The procedure for converting a non-mucosally-active microbial antigen to a mucosally-active, vaccine is detailed in the examples below.

Immunizing can mean oral administration, inhalation, enteral, feeding or inoculation by intravenous injection.

The phrase “antibodies effective to ameliorate or clear Borellia infection” means that the antibodies induce protective immunity through the endogenous immune system in an organism in vivo, such that the infection is fought through the organism's natural immune process.

“High affinity” for an IgG antibody refers to an antibody having a KD of 10⁻⁸ M or less; or 10⁻⁹ M or less; or 10⁻¹⁰ M or less. However, “high affinity” binding can vary for other antibody isotypes. For example, “high affinity” binding for an IgM isotype refers to an antibody having a KD of 10⁻⁷ M or less; or 10⁻⁸ M or less.

### II. EXAMPLES

The following examples are illustrative in nature and are in no way intended to be limiting.

Provided herein is a plant-expressed fusion protein comprising cholera toxin B subunit (CTB) adjuvant fused to a Borrelia outer surface protein A (OspA) protein, polypeptide or peptide fragment thereof. In one aspect of the present disclosure, a codon-optimized nucleic acid sequence having at least 90% sequence identity to SEQ ID NO: 1, encoding a cholera toxin B subunit (CTB) adjuvant fused to an outer surface protein A (OspA) protein, polypeptide or peptide fragment thereof, is provided. In one aspect, an amino acid sequence having at least 90% sequence identity to the sequence identified by (SEQ ID NO: 2) is provided. In one aspect, an amino acid sequence having at least 90% sequence identity to the sequence identified by (SEQ ID NO: 2) and encoded by the codon-optimized nucleic acid sequence having at least 90% sequence identity to SEQ ID NO: 1 is provided.

The present disclosure generally relates to the transformation, selection and generation of pure rice seed stock expressing high levels of recombinant microbial protein with good growth performance in the open field. With the compositions and methods disclosed herein, a sufficient amount of rice grain has been produced for animal studies of immunization against Borrelia infection. Rice flour generated from the Osp protein expressing rice was administered orally to laboratory mice. Serum antibody titers were optimized and vaccine effectiveness determined. Vaccines were found to 1) protect mice from infection by bites of Borrelia burgdorferi-infected ticks, and 2) reduce or eliminate infection in ticks feeding on an immunized host. An oral rOspA vaccine formulation described herein was used in a field study to determine whether immunization 1) reduced B. burgdorferi infection rates in rodent reservoir animals, especially mice, and 2) significantly reduced infection rates in tick vector populations.

Previous studies conducted with rice-derived OspA protein which was orally administered failed to generate protective antibodies or to protect mice. In contrast, protective antibodies have been generated using the exemplary CTB.OspA fusion protein as provided herein.

### Example 1

### Production of OspA Rice Grain

Field-grown homozygous lines which expressed high levels of rOspA were obtained. A total of 3350 grams of pure stock seed was produced. To obtain additional grains that stably express high levels of rOspA, the transgenic lines were grown in a summer nursery in Kansas and in a winter nursery in the US Virgin Islands. Table 1 shows line selection and grain production from the harvest of the latest seasons (2009 and 2010 summer season, Junction City, Kans.). Based on comparisons to known standards, the OspA expression level is estimated to be about 1 g/kg rice flour. The estimated expression level of each event and over two years of growth is shown in Table 1.

TABLE 1
Line selection and production of transgenic
rice grain expressing OspA.

| | | Panicle/ | Grain | OspA Exp |
|---|---|---|---|---|
| Events | Season | Plants | (lb) | (g/kg flour) |
| VB15-5 | 2009 | 50 | 9.6 | ~1 g/kg |
| | 2010 | 50 | 592 | ~1 g/kg |
| VB15-111 | 2009 | 50 | 400 | ~1 g/kg |
| | 2010 | 50 | 381 | ~1 g/kg |

Exp = expression measured based on gel analysis

Over these seasons, a total of 1382.6 lbs. of rice grain expressing OspA were produced. 50 plants were individually harvested from each season to keep the lines pure. A breeding program was initiated to transfer the OspA gene from the Taipei 309 genetic background to elite rice lines, with two major breeding goals: (1) increased grain yield, and (2) generation of a sustainable yield.

A total of 56 germplasms were collected and screened based on field performance, such as flowering date, synchronization in flowering, maturation date, plant height, set seed, plant type and rice blast resistance in a rice nursery located in Junction City, Kans. Further screening was carried out in the lab for the following characteristics: seed setting rate, filled seeds per panicle, harvest index, 1000 grain weight and grain yield. Fourteen rice lines were chosen as crossing parents in a crossing program. In general, the 14 rice lines possess desirable agronomic traits at the Junction City plant nursery, such as early maturation, high seed setting rate, harvest index, grain yield and resistance to infection by rice blast pathogens. All the selected lines matured in <135 days after planting, except for 4641, which needed about 145 days to mature. The entries generally produce grain yield over 6000 lbs/acre (6818 kg/ha). Ten of 14 lines possess less than 95 cm plant height, which reduces the probability of lodging in windy days, especially after the grain filling stage to maturity (Table 2).

TABLE 2
Elite germplasms screened and identified as suitable for cross-breeding.

| | | Plant | | Filled | Seed | 1000 | Grain | |
|---|---|---|---|---|---|---|---|---|
| | Maturation | height | Harvest | seeds/ | setting | grain | yield: | Rice |
| Rice lines | date (days) | cm | index | panicle | rate | weight | lbs/acre | blast |
| ZHE 16 | 122 | 82.3 | 0.5510 | 88.9 | 96.53% | 27.94 | 7332.6 | No |
| ZHE 73 | 122 | 79.8 | 0.5503 | 79.3 | 92.97% | 28.02 | 8349.0 | No |
| Minkezao | 123 | 85.1 | 0.5385 | 104.1 | 95.19% | 27.68 | 7215.7 | No |
| Chunjiangzao | 122 | 87.0 | 0.4986 | 90.0 | 87.29% | 27.44 | NA | No |
| Zhong 86 | 125 | 94.7 | 0.5470 | 107.4 | 97.28% | 23.03 | NA | No |
| Neptune | 133 | 93.0 | 0.4986 | 128.0 | 87.06% | 26.97 | 7635.9 | No |
| Cybonnet | 130 | 101.0 | 0.4770 | 115.6 | 92.64% | 23.55 | 7651.2 | No |
| Japan 92 | 132 | 97.2 | 0.5424 | 110.6 | 95.65% | 25.65 | 6417.7 | No |
| Luhongzao | 125 | 85.9 | 0.4695 | 80.0 | 95.58% | 30.26 | 6388.8 | No |
| You-lb | 123 | 80.8 | 0.5003 | 78.8 | 97.52% | 23.36 | NA | No |
| G5830 | 125 | 92.7 | 0.4995 | 97.6 | 83.22% | 26.19 | 5314.6 | No |
| 4641 | 145 | 106.7 | 0.4867 | 104.8 | 92.15% | 26.47 | 7764.5 | No |
| Indica 2 | 125 | 106.7 | 0.4044 | 86.9 | 92.67% | 24.57 | 5955.9 | No |
| Indica 7 | 125 | 80.3 | 0.4838 | 86.5 | 91.78% | 25.34 | 6341.9 | No |

### Example 2

### Expression of Cholera Toxin B Subunit in Rice Grain

US Patent Application Publication 20110117131 (Huang, et al.), incorporated by reference herein in its entirety, describes the generation of transgenic rice expressing recombinant OspA (rOspA) protein for the use as a vaccine. To date, it remains unclear whether linkage of CTB to OspA produced in plant cells is required to make OspA mucosally active.

Gene Construct and Transformation—

To obtain high expression levels of recombinant CTB (rCTB) in rice grains, the mature CTB protein amino acid sequence (UniProt accession number P01556) was back-translated into a nucleotide sequence with the codons optimized towards the codon-usage preference of rice genes, while the internal repeats and other features that might affect mRNA stability or translation efficiency were not altered. The entire nucleotide sequence was synthesized by the company DNA2.0, and then ligated in frame into a backbone plasmid vector called pAPI405, which contains the rice seed storage protein glutelin 1 gene (GenBank accession no. Y00687) promoter (Gt1), signal peptide encoding sequence, and the terminator of the nopaline synthase (nos) gene of the T-DNA in Agrobacterium tumefaciens. The resulting plasmid was verified by sequencing in both orientations, and designated as pVB45. The linear expression cassette of DNA fragments comprising the region from promoter to terminator (without the backbone plasmid sequence) of VB45 plasmid was liberated with EcoRI and HindlII double digestion and used for microprojectile bombardment-mediated transformation of embryonic calli induced from the mature seeds of cultivar Bengal (Oryza sativa, subsp. Japonica). Seventy one transgenic rice plants containing the CTB transgene were then identified by PCR using primers specific to the nucleotides encoding CTB, and then grown in a greenhouse for seed production.

Expression Screening Analysis of Transgenic Seeds—

Out of 71 transgenic plants, 37 were able to produce seeds (R1). To identify transgenic plants expressing rCTB, seed proteins were extracted from eight pooled R1 seeds of each individual plant in PBS buffer, pH 7.4, at RT for 30 min. Two microliters of the pooled crude protein extract from each transgenic event were spotted onto a nitrocellulose membrane, and nine positive transgenic plants expressing CTB were identified by immuno dot-blot expression analysis. The Western blot analysis further demonstrated that recombinant CTB cross-reacting specifically with anti-CTB antibody was present in the crude protein extracts of positive transgenic rice seeds but absent in wild-type rice seed protein extracts (data not shown). Three bands were demonstrated by Western blot assay. The molecular size of the bottom band is shown to be the same as that of commercial recombinant CTB. A band right above the bottom band represents rCTB with post-translational modification, most likely N-linked glycosylation, and the uppermost band seems to be a dimer form of rCTB. Two independent transgenic lines, VB45-353 and VB45-360, with the highest level expression of rCTB were then selected for propagation.

To select the homozygous lines expressing rCTB, over 100 R1 seeds of each selected transgenic rice line were grown to the next generation. For each R1 line, over 20 R2 seeds were assayed by immuno-dot-blot to monitor the genetic segregation of rCTB expression. Seed total soluble proteins were extracted from twelve seeds of each transgenic line with 250 μl/seed of PBS buffer, pH 7.4 at RT for 10 min followed by centrifugation. Then, 3 μl of protein extract from each seed was spotted onto a nitrocellulose membrane and probed with anti-CTB antibody (Sigma) (data not shown). Wild-type Bengal seed protein extract was used as a control. Four lines with all R2 seeds shown as positive were found to be homozygous: VB45-360-143, VB45-353-141, VB45-353-142, and VB45-353-144. Two lines, VB45-353-41 and VB45-360-54 were homozygous negative, and VB45-353-58 was heterozygous. Overall, more than forty homozygous lines were selected, and the expression level in R2 rice grain was estimated to be 0.2% seed dry weight.

Functional Characterization of Rice-Expressed rCTB—

To assess whether rice-derived CTB forms a pentamer, Western blot analysis of rCTB under non-reducing and non-boiling conditions was performed (data not shown). The majority of rice-derived rCTB was found to be present as proteins of approximately 60 kDa, indicating that rCTB formed a pentamer.

To further test the biological activity of rice-derived CTB, a GM1 (monosialotetrahexosylganglioside) binding assay of rCTB in rice seed protein extract by GM1-ELISA was carried out. No GM1 binding activity was found in wild-type rice seed protein extract, whereas the transgenic rice seed extract demonstrated levels of GM1 binding activity similar to that of control recombinant CTB protein (data not shown). Furthermore, rice-derived rCTB also showed a dose-dependent GM1 binding activity similar to control CTB (data not shown).

### Example 3

### Immunogenicity of Rice rOspA in C3H/HeJ Mice

C3H/HeJ mice, a strain that is highly susceptible to B. burgdorferi infection, were used for all immunizations. The rOspA immunogen was purified from rice. The antigen in PBS was prepared with an equal volume of alum adjuvant (Imject, Pierce) to yield a vaccine with 12.5 mg of rOspA per 100 ml dose, delivered intraperitoneally. A primary immunization was given, followed by two booster immunizations. A commercially available canine OspA vaccine (Recombitek Lyme, Merial) was used as a positive control at the same immunizing doses as per the manufacturer's instructions. Blood samples for serum preparation were collected by facial artery/vein plexus bleeding using a 5-mm lancet while mice were under isoflurane anesthesia. The ability of mice to produce anti-OspA antibody was determined by immunoblot using whole cell antigen of B. burgdorferi strain B31 (Viramed Biotech AG, Germany). Mouse antibodies that bound B. burgdorferi antigens were detected with phosphatase-labelled goat-anti mouse IgG (H+L) (KPL, Inc.), diluted 1:1000 in blocking buffer. This demonstrated that recombinant OspA purified from transgenic rice is immunogenic in mice and elicits a high-titered response after three injected doses.

Efficacy of rice rOspA vaccine in protecting C3H/HeJ mice from cultured B. burgdorferi administered by subcutaneous inoculation—Four weeks after the final boost immunization, all mice were challenged with a low passage strain of B. burgdorferi (B31 clone A3) harvested in mid-logrithmic phase of growth in BSKII medium. One group of animals received a lower challenge inoculum consisting of 2×10³ bacteria/mouse and another group received a higher inoculum of 2×10⁴ spirochetes. Two weeks after inoculation with B. burgdorferi, the infection status of each animal was assessed by culture of a skin biopsy sample (˜25 mm²). Four weeks after inoculation, infection status of internal organs was assessed, by culture of skin and by culture of heart and bladder, target organs of B. burgdorferi. Cultures were read by dark field microscopy after 10 days of incubation at 34° C. under microaerophilic conditions. If negative at 10 days, they were examined a second time at 3.5 weeks. Potential seroreactivity with non-OspA antigens after challenge was assessed by IgG immunoblots against whole cell antigens and by IgG antibodies to recombinant VISE, a highly sensitive and specific antigen that is recognized early in the course of B. burgdorferi infection (Viramed Biotech). Seroconversion was studied using a 1:100 dilution of serum.

Rice rOspA protected all mice from B. burgdorferi administered by needle at a dose of up to 2×10⁴ organisms per animal. This protection was statistically significant (p=0.0001). No evidence of seroconversion was found after cultured organism challenge, and all cultures read at both time points were negative for rice rOspA immunized mice. The culture of B. burgdorferi used for challenge was highly infectious, since 75% of unimmunized animals were culture positive. All culture-positive animals seroconverted, whereas culture-negative ones did not.

TABLE 3
Ability of rice rOspA to protect mice from B. burgdorferi infection

Proportion of mice positive by culture

| | Low dose | High dose | Combined low and |
|---|---|---|---|
| Immunogen | challenge | challenge | high dose results |
| Rice rOspA | 0/6 | 0/7 | 0/13* |
| Merial vaccine | 0/2 | 0/2 | 0/4 |
| None | 6/10 | 9/10 | 15/20* |

*p = 0.0001 by Fisher's exact test, 2-tailed.

Mice immunized with rice-derived OspA via injection were protected from needle-inoculated, culture-grown B. burgdorferi. To examine whether the mice were subsequently protected from challenge via infected ticks, further studies on these immunized mice were carried out.

Efficacy of rice rOspA vaccine in protecting C3H/HeJ mice challenged with Ixodes scapularis ticks infected with B. burgdorferi. Since rOspA immunized mice were protected from challenge by cultured B. burgdorferi administered by needle, they were challenged a second time in a small pilot experiment to see whether they also would be protected against infection by tick bites. Although no evidence was found of Borrelia infection after needle challenge, administration of cultured bacteria theoretically could have resulted in antigenic stimulation of the mice (even though no evidence of production of antibodies other than anti-OspA was seen by immunoblotting). In view of this limitation, a pilot study was conducted with mice that had received the lowest dose of challenge organisms (see Table 3).

Colony-raised I. scapularis nymphs infected with B. burgdorferi strain B31 were used for tick challenges. The infection rate in this colony is about 90%, determined by culture of ticks in BSK II medium. Five B31-infected nymphs were placed on the necks of each rOspA immunized mouse and controls (Swiss Webster outbred mice) while the animals were under isoflurane anesthesia. The average number of nymphs that attached and were recovered for analysis after feeding was 2.9 per mouse Table 4. (Some ticks may be groomed off by mice and even eaten).

TABLE 4
Ability of rice rOspA to protect mice
and clear spirochetes from ticks

| | | Tick Challenge | | |
|---|---|---|---|---|
| | Mouse | Mouse | Proportion of | LA2-Equivalent |
| Immunogen | # | infection | infected tick | antibody (ng/ml) |
| Rice rOspA | M210 | No | 0/1 | 7548 |
| | M221 | No | 5/5 | 4611 |
| | M224 | No | 1/3 | 8384 |
| | M225 | Yes | 3/3 | 3166 |
| | M No Tag | No | 3/3 | NA |
| None | M1 | Yes | 3/3 | NA |
| | M2 | Yes | 2/2 | NA |
| | M3 | Yes | 3/3 | NA |

NA—serum sample not available.

As can be seen from Table 4, four of the five mice were protected and remained culture negative. On the other hand, only a few ticks were cleared of spirochetes, namely one tick from mouse M210 and two ticks from M224. The challenge was robust because all control animals became infected by tick bites and all recovered ticks were shown to be infected by culture in BSK II medium.

Correlation of Mouse Infection, Tick Infection and LA2-Equivalent Antibody Level—

A monoclonal antibody designated LA2 defines the major protective epitope on OspA. Serum antibody responses after OspA immunization may be analyzed for the amount of IgG that competes for binding to the LA2 site on OspA. This value, designated LA2-equivalent antibody, is highly correlated with a protective antibody response from previous studies. The LA2-equivalent titers in mice that were immunized with rice rOspA and challenge by tick bites were examined by competitive ELISA to learn whether the LA2-equivalent antibody titers in serum of mice M210 and M224 were higher than the levels in other mice.

For the LA2 competitive ELISA, OspA produced in Esherichia coli and derived from the Merial vaccine for dogs was used to coat plates. This OspA, designated mOspA, was dialyzed against PBS and stored at −20° C. until use. Microwell plates (Fisher 442404) were coated with 100 μl/well of mOspA after dilution to 100 ng/ml in coating buffer (90 mM NaHCO₃, 60 mM Na₂CO₃, pH 9.6). The plate was incubated at 4° C. overnight and then washed five times with TBS-T buffer (10 mM Tris, 140 mM NaCl, 2.7 mM KCl, 0.05% Tween 20, pH 7.4) and then blocked with 250 μl/well of blocking buffer (TBS-T buffer plus 1% BSA) at 37° C. for 60 mins. Purified mouse monoclonal IgG antibody LA2 was used as the standard. LA2 and serum samples were diluted in blocking buffer. The LA2 standard (500 ng/ml) was diluted by serial two-fold dilution to 31.25 ng/ml. Serum samples were diluted 25-fold in blocking buffer. The LA2 standards and serum samples were run in duplicate, 100 μl/well applied to each well. The plate was then incubated at 37° C. for 60 mins, washed, and biotinylated LA2 antibody (diluted to 100 ng/ml) was then applied to each well containing standard or serum samples. The plate was incubated at 37° C. again for 60 mins, washed and peroxidase-labeled streptavidin (KPL 14-30-00) diluted to 1 μg/ml in blocking buffer was then added to each well at 100 μl/well. The plate was again incubated at 37° C. for 60 mins and then washed. One hundred microliters of peroxidase substrate (SureBlue Reserve TMB Microwell, KPL 53-00-01) was then added to each well and incubated at room temperature for 15 min before 100 μl of stop solution (TMB blueSTOP Solution, KPL 50-85-30) was added. Absorbance of individual wells was evaluated at 630 nm.

To determine the concentration of LA2 equivalent antibody in serum samples, concentrations of standard were log₁₀-converted. The mean absorbance values for each concentration of LA2 were used to establish a linear regression relationship between OD readings and concentrations of the LA2 standard. An estimate was made regarding unknown samples based on the linear relationship between OD readings and LA2 standards. To adjust for the serum effect, background levels of negative serum samples were subtracted from each serum sample. After factoring in a dilution factor, the concentration of LA2 equivalent antibody in serum samples was determined and listed in Table 4. There was no serum available for mouse number “no tag”; therefore, data are not available.

As can be seen from Table 4, LA2 equivalent antibody level was lowest for mouse M225. This mouse was infected via tick challenge although it was protected when challenged with cultured B. burgdorferi administered by subcutaneous inoculation. It is possible that the serum titer needed to protect mice is different depending on how mice are challenged. Mouse M221 has a higher LA2 antibody titer than M225. This mouse was protected when tick-challenged, but all five ticks remained infected. The two mice from which ticks were partially cleared of B. burgdorferi have the highest LA2 equivalent antibody levels. It has been shown by others that the levels needed to clear ticks of spirochete infection are much higher than that needed to protect against spirochete transmission. In these studies the LA2 antibody level is positively correlated with mouse protection and tick clearance, consistent with observations in the literature. The information in Table 4 is useful as it can serve as a guide for oral immunization studies. When well-validated in such protocols, LA2 equivalent titers may define target antibody levels that must be achieved for successful oral immunization.

### Example 4

### Feeding Bait Preparation

Feeding Bait Preparation—

Feeding baits were prepared as needed during the period of oral immunization. A first type of the baits is called 50% OspA bait because it contains 50% transgenic OspA rice flour. The composition of the 50% OspA bait is 50% transgenic OspA rice flour, 30% peanut butter, 10% oats and 10% paraffin to mold the bait preparation. To prepare the bait, transgenic rice grain expressing OspA was ground into rice flour and stored at room temperature. On the day of making bait, 100 grams of transgenic rice flour was placed in a 500 ml beaker, along with 20 grams of oats, 20 grams of paraffin, and 60 grams of peanut butter. This beaker was then placed inside a 1000 ml beaker which contained 400 ml of water. Both beakers were then placed on a heat block and the heat adjusted to melt both the peanut butter and paraffin. Upon melting the peanut butter and paraffin, 100 grams of flour was then placed into the beaker to effectively mix all four components. This mixture was then placed into a mini-ice cube tray to form individual baits of approximately 5 grams/bait upon cooling at 4° C. The 50% OspA baits were used for groups 1 and 2.

A second type of the feeding bait is called control bait because it contains non-transgenic rice flour. The same method to make 50% OspA baits was used except that control (non-transgenic) rice flour was used. Group 4 mice were fed with control bait.

### Example 5

### Oral Immunization in C3H/HeJ Mice with Bait Containing Rice OspA

Oral Immunization in C3H/HeJ mice with bait containing rice-derived OspA: Female C3H/HeJ mice (four weeks) were purchased from Jackson Labs. C3H/HeJ mice were used for the same reason as they were used for injection study (i.e., sensitivity to spirochete infection and the development of spirochete-induced pathology). Oral immunization was initiated when the mice were six weeks old. Twenty mice were randomly assigned to four immunization groups. Each mouse was housed in a separate isocage to monitor invidual bait consumption. Bedding material was removed and replaced with a cardboard paper cage liner to monitor daily consumption. Water was supplied as needed. Each morning during the immunization trial, remnants of bait material were weighed to determine the amount of the bait consumed per day. Then new bait was placed in the cage. This process was repeated following the immunization scheme stated above. When immunization was complete, five mice of the same group were placed into a regular holding cage. Regular mouse feed and water were supplied as needed and bedding materials were placed.

There were five mice in each experimental group. Group 1 received 50% transgenic rOspA flour with no adjuvant; group 2 received 50% rOspA flour and 70 μg CTB, administered by gavage on each day of vaccine baiting; group 3 received 50% wild-type rice flour with 70 μg CTB; and group 4 received 50% wild-type rice flour with no adjuvant. The remainder of the bait comprised of 30% peanut butter, 10% oats and 10% paraffin to mold the bait preparation.

Table 5 below shows immunization scheme of 4 groups.

| | Treatment | Mice/ | CTB | OspA flour |
|---|---|---|---|---|
| Group | groups | group | (ug/dose) | in Bait |
| 1 | OspA flour | 5 | 0 | 50% |
| 2 | OspA flour + CTB | 5 | 70 | 50% |
| 3 | CTB only | 5 | 70 | 50% |
| 4 | Regular rice flour | 5 | 0 | 0 |

C3/HeJ mice were allowed to feed ad libitum control or rOspA rice flour for 14 days, followed by a seven day rest period on normal mouse chow, and boosted daily for seven days before infected tick challenge. For treatment groups immunized with CTB as an adjuvant, 70 μg CTB (70 μg/dose) in a 50 μl volume was delivered by oral gavage on the days where rOspA was present in the bait.

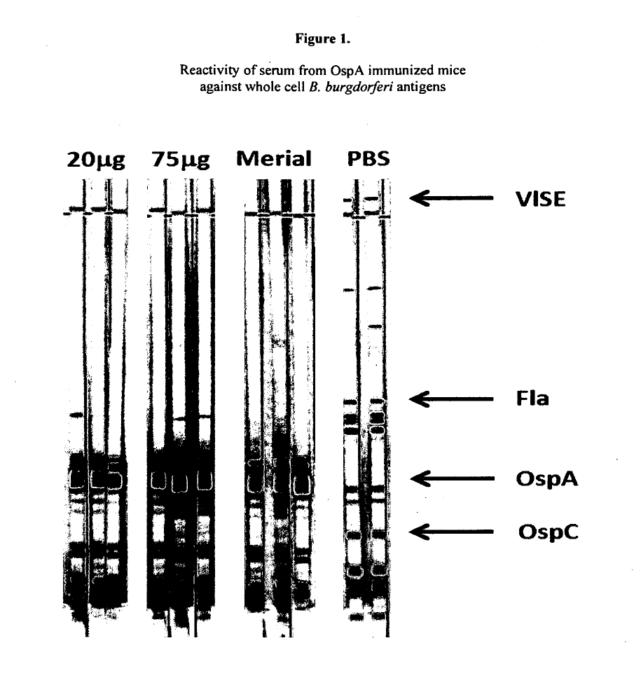

FIG. 1 demonstrates the average bait consumption in groups 1, 2 and 4 (Table 5). There is no statistical difference in bait consumption among groups, indicating that the feeding of rCTB had no effect on the appetites of individual mice. The mice ate, on average, 4 g of bait per day or 2 grams of recombinant flour. Since the expression level of OspA was estimated to be about 1 mg/gram flour, mice in groups 1 and 2 were immunized with approximately 2 mg of OspA daily.

### Example 6

### Bleeding and ELISA Analysis of Anti-OspA Antibody in Serum

All mice were bled on day 16 from first immunization series, and subsequently three days prior to infected tick challenge during the booster immunization week to harvest serum. Total anti-OspA antibody titers were measured by ELISA using Merial vaccine OspA to coat plates. Two-fold dilutions were made, starting at a 1:100 dilution of serum (Table 6). The control group (group 4) gave a reciprocal titer of 400 as background. The titer for groups 1 and 2 ranged from 3,200 to 25,600. There was no difference between groups with or without CTB. As expected, the CTB group has the same titer as the control group.

Three days after bleeding, mice were challenged with infected ticks. Briefly, each mouse received five infected nymphal ticks (B31 strain) under isoflurane anesthesia and then placed in individual cages to monitor tick feeding. Infected Ixodes scapularis ticks fed to repletion over a 4 day period. At this point individual ticks were collected per animal and all animals were returned to gang housing per immunization group. On average, 2.4 to 3.4 ticks fed to repletion on each mouse (Table 6) and infectivity of ticks averaged 90%, typical of the infected tick colony. Table 6 shows that CTB-adjuvanted rOspA-bait fails to protect C3H/HeJ mice or to clear ticks from B. burgdorferi infection.

TABLE 6
Serum anti-OspA titer and tick challenge

| | | | Avg. # ticks | Endpoint anti- |
|---|---|---|---|---|
| Group | ID | Bb | fed/mouse | Osp titer |
| OspA bait | 681 | + | 2.4 | 1:3,200 |
| Group 1 | 682 | + | | 1:12,800 |
| | 683 | + | | 1:12,800 |
| | 684 | − | | 1:25,600 |
| | 685 | + | | 1:12,800 |
| OspA bait + CTB | 686 | + | 2.6 | 1:12,800 |
| Group 2 | 687 | + | | 1:12,800 |
| | 688 | + | | 1:25,600 |
| | 689 | − | | 1:12,800 |
| | 690 | + | | 1:12,800 |
| Control bait | 691 | + | 3.4 | 1:400 |
| Group 4 | 692 | + | | 1:400 |
| | 693 | + | | 1:400 |
| | 694 | + | | 1:400 |
| | 695 | + | | 1:400 |
| CTB only | 676 | + | 2.2 | 1:800 |
| Group 3 | 677 | + | | 1:800 |
| | 678 | + | | 1:200 |
| | 679 | + | | 1:400 |
| | 680 | + | | 1:400 |

“+” in the “Bb” column means the mice were infected; “−” means the mice were not infected (or protected from prior infection).
Anti-OspA titers in mouse serum represent total IgG, rather than LA-2 equivalent IgG titers.

Out of the 10 mice fed with OspA flour (either with or without CTB; groups 1 and 2), only two mice were protected, compared with no protection in the bait only control and CTB-alone groups. A 20% protection rate is considered very low. All ticks collected remained infected as demonstrated by positive cultures in BSK II medium. It was anticipated that at least 80% of mice would be protected and more than 50% of ticks cleared of spirochete infection, based on related literature. Comparing the present immunization protocol in the literature, two major differences between protocols are evident. First was the immunization period. The present immunization period is shorter (first dose to last dose is 28 days and first dose to tick challenge is 31 days), and the hope was that this protocol would be more efficient for specific field studies. The protocol in the literature was much longer (first dose to last dose is 46 or 55 days and first dose to tick challenge is 67 days). The second difference was amount of OspA per dose. As stated before, mice in the present protocol, on average, consumed 4 grams of feeding bait or 2 gram rice flour which contains about 2 mg of OspA. The protocol in the literature used three different concentrations, but the 100 mg lyophilized E. coli gave the best results. Analysis shows that about 5 mg of OspA is present in 100 mg lyophilized E. coli powder. Thus, longer immunization times as well as a higher antigen level might be expected to stimulate appropriate antibody levels for protection.

Based on the previous oral immunization study, the immunization period was extended, dosage levels were increased and LA2 equivalent antibody was measured as the study progressed. Thus, the dosage of rOspA rice flour was increased from 50% to 95% by mass in the baited formulation, and the duration of feeding was extended to nine weeks. The study design and immunization scheme based on 6 groups are outlined in Table 7, below.

TABLE 7
Study design for increased rOspA dose and
duration of oral delivery to C3H/HeJ mice.

| | Treatment | Mice/ | Dosing | CTB | OspA flour |
|---|---|---|---|---|---|
| Group | groups | group | Frequency | (ug/g bait) | in Bait |
| 1 | OspA flour | 5 | 5 d/w | 0 | 50% |
| 2 | OspA flour + | 5 | 5 d/w | 20 | 50% |
| | CTB | | | | |
| 3 | OspA flour | 5 | 1 d/w | 0 | 50% |
| 4 | Regular rice | 5 | 5 d/w | 0 | 0 |
| | flour | | | | |
| 5 | OspA flour | 5 | 4 d/w | 0 | 50% |
| 6 | OspA flour | 5 | 4 d/w | 0 | 95% |

Each group had five mice. All groups were immunized for nine weeks. The first five groups were immunized and group 6 was added two weeks later. Group 1 is being immunized with bait 5 days/week. The bait contains 50% transgenic OspA rice flour, 30% peanut butter, 10% oats and 10% paraffin (see details supra).

Group 2 was immunized with the same bait as group 1, except that the bait contains cholera toxin B subunit (CTB) at 20 μg/gram bait. CTB has been shown to act as a mucosal adjuvant. The addition of CTB is predicted to induce a greater immune response to rOspA.

Group 3 was immunized with the same bait as in group 1, but delivered one day per week to avoid a potential over-dose in group 1 that might induce oral tolerance.

Group 4 served as a negative control by being fed with non-transgenic rice flour 5 days/week. The bait contains 50% non-transgenic rice flour, 30% peanut butter, 10% oats and 10% paraffin (see details in section on bait preparation).

Group 5 was immunized with the same bait as in group 1, but delivered only four days every three weeks. This immunization scheme was used by another group delivering OspA made in E. coli which generated protective immunity. The same immunization scheme was tested herein to determine if rice-derived OspA can generate similar protective immunity.

Group 6 was added later and utilizes feeding bait 4 days/week. This bait contained 95% transgenic OspA rice flour plus 5% peanut butter, a formulation which permits immunization with twice the amount of rOspA.

Feeding Bait Preparation—

In this example, four types of mouse feeding baits were. As in earlier studies, baits were prepared to deliver 5 grams per day. Baits were prepared as needed during the period of oral immunization.

The first type of the bait is called 50% OspA bait because it contains 50% transgenic OspA rice flour. The composition of the 50% OspA bait is 50% transgenic OspA rice flour, 30% peanut butter, 10% oats and 10% paraffin. To prepare the bait, transgenic rice grain expressing OspA was ground into rice flour and stored at 4° C. until use. 100 grams of transgenic rice flour was placed in a beaker which was then placed in a 50° C. incubator to pre-warm the flour. After 60 mins incubation, 20 grams of oats, 20 grams of paraffin, and 60 grams of peanut butter were added to the flour in a 800 ml beaker. The 800 ml beaker was then placed inside a 1000 ml beaker which contained about 300 ml of water. Both beakers were then placed on a heat block to melt the peanut butter and paraffin. Upon melting peanut butter and paraffin, the smaller beaker was taken out and the contents were allowed to cool down to 60° C. or slightly below. The reason to cool the content to less than 60° C. is that the thermo-transition temperature of OspA is 59° C. When cooled, 100 grams of flour (50° C.) was added. The four components were then mixed quickly and completely. This mixture was placed into a mini-ice cube tray to form individual 5 g baits at 4° C. Approximately 40 feeding baits were made at a time using this method. The 50% OspA baits were used for groups 1, 3 and 5.

The second type of the feeding bait is called CTB bait because it contains 20 μg/gram of cholera toxin B subunit (CTB). The same method was used to make CTB baits except that prior to adding the 100 grams of OspA flour, 4 mg of CTB (Sigma) was added to the mixture (cooled down to below 60° C.) of three components (peanut butter, oats and paraffin) and mixed well. The CTB baits were then used to immunize group 2 mice.

The third type of the feeding bait is called control bait because it contains control (non-transgenic) rice flour. The same method was used to make this bait as described above and group 4 mice were fed with control bait.

The fourth type of the feeding bait is called 95% OspA bait because it contains 95% transgenic OspA rice flour. To prepare 95% OspA bait, 190 grams of transgenic rice flour were thoroughly mixed with 10 grams of peanut butter. Then 100 ml of water was added to make a dough-like mixture which was placed into mini-ice cube trays and then frozen −20° C. After solidification, individual cubes (about 40 cubes) were then placed on absorption paper within a laminar flow hood. The individual cubes/baits were left overnight night within the hood and checked for moisture content by weighing the entire batch of cubes, which should be less than 205 grams total. The drying and desiccation process was continued until the total weight was less than 205 grams. The baits were then stored at 4° C. until use.

Oral Immunization of C3H/HeJ Mice with Bait Containing Rice OspA—

Again, female C3H/HeJ mice were purchased from Jackson Labs. Oral immunization was initiated when the mice were 6 weeks old. 30 mice were randomly assigned to six immunization groups. Each mouse was housed individually in an isocage containing a cardboard paper liner to monitor bait consumption. Bait was then placed inside the isocage and water was supplied as needed. Each morning of the immunization protocol, remnants of bait were weighed to determine the daily amount of the bait consumption. New bait was then placed in the isocage. This process was repeated for the number of days indicated in Table 7 for each group. When immunization was complete for the week, either 1, 4 or 5 days, all five mice of the same group were placed into a regular cage for maintenance. Feed and water were supplied as needed and an entertainment roller and roll were provided. The mice were fed for 9 weeks or until LA2 equivalent antibody in mouse serum reaches 9000 ng/ml of serum.

Mice tend to consume more bait on the first day when transferred from regular cages to isocage. Thus mice in group 3 which were immunized once per week consumed more bait. All other groups consumed similar amounts, approximating 4 grams/day/mouse and similar to the previous oral immunization study (FIG. 1).

Immune Response to Oral Immunization in Mice—

The first bleeding took place on day 21 after initial dosing. Serum samples were collected by facial artery/vein plexus bleeding using a 5-mm lancet while mice were under isoflurane anesthesia. Serum samples were stored at −80° C. until analyzed.

Competitive ELISA to determine the level of LA2 equivalent antibody was carried out with serum samples collected on day 21. No detectable LA2 equivalent antibody was noted among all groups at day 21. As the experiment progresses, LA2 equivalent antibody in mouse serum will be further monitored.

### Example 7

### Generation of a Recombinant CTB.OspA Fusion Protein to Increase the Oral Immunogenicity of OspA Antigen

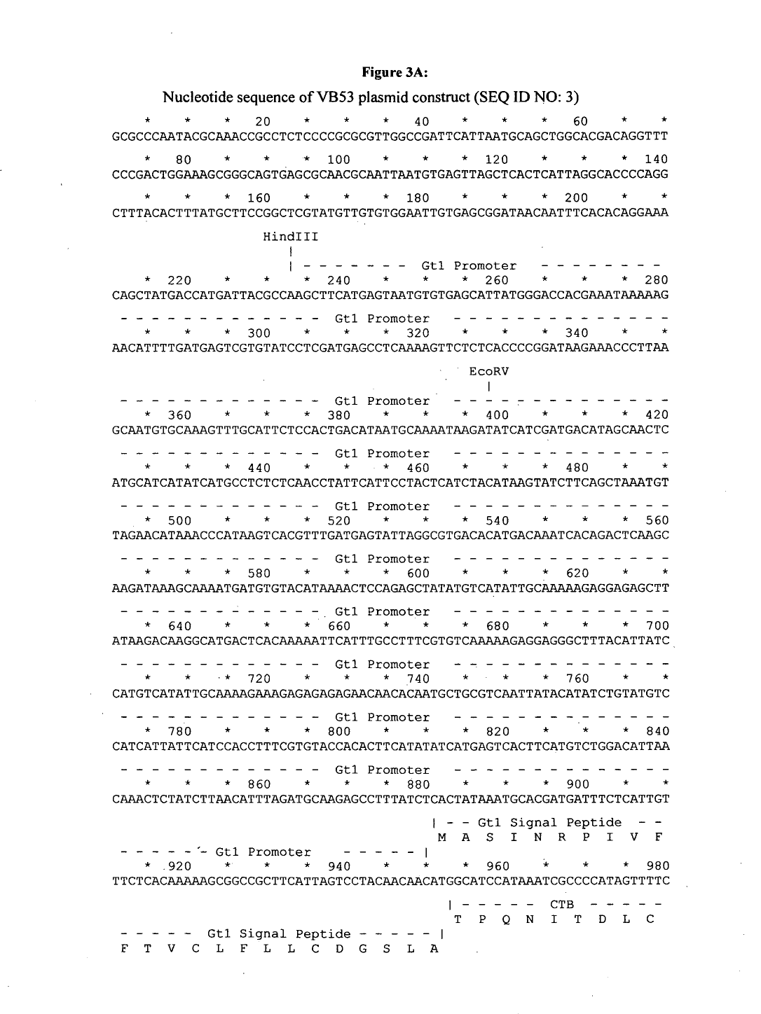

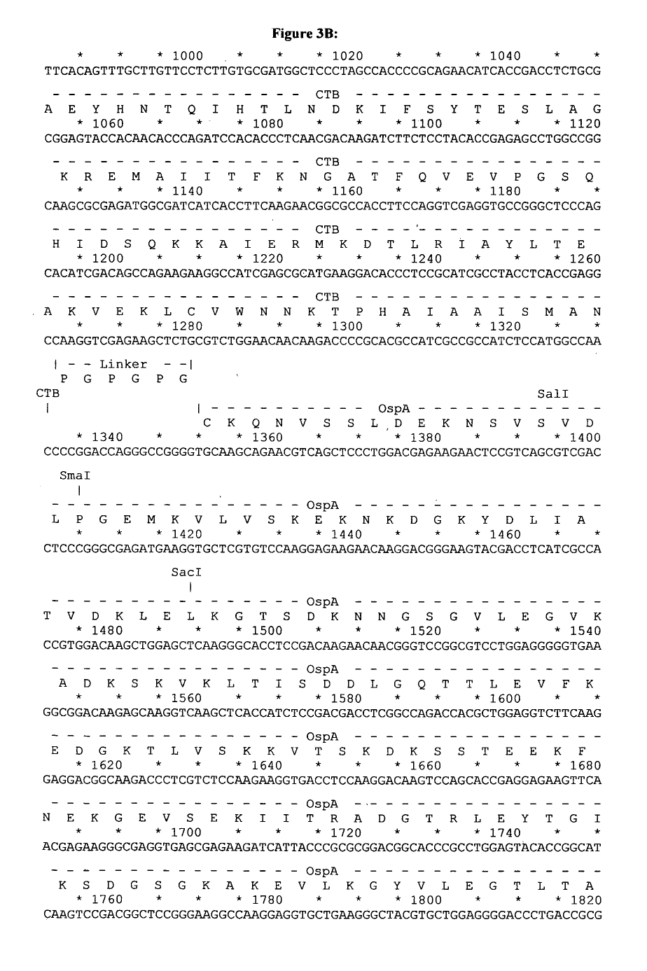

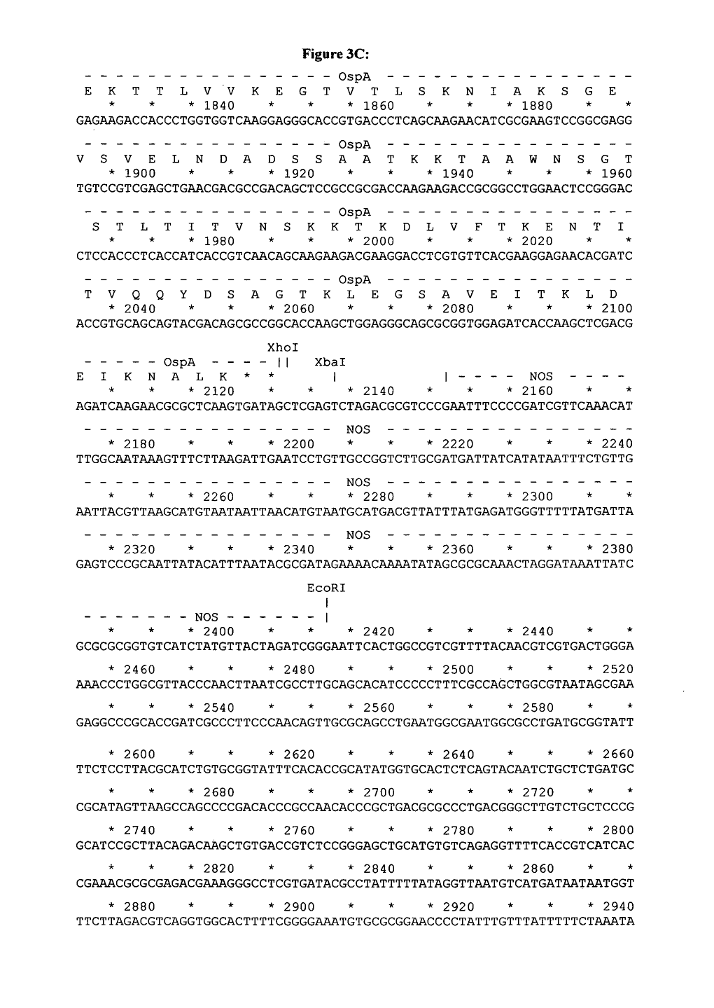

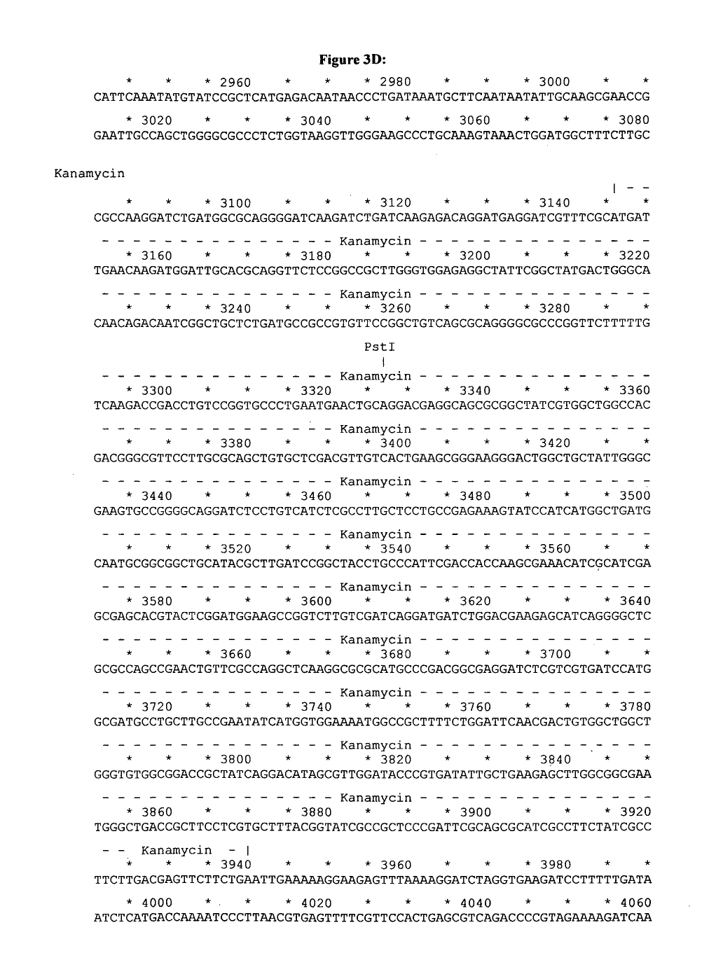

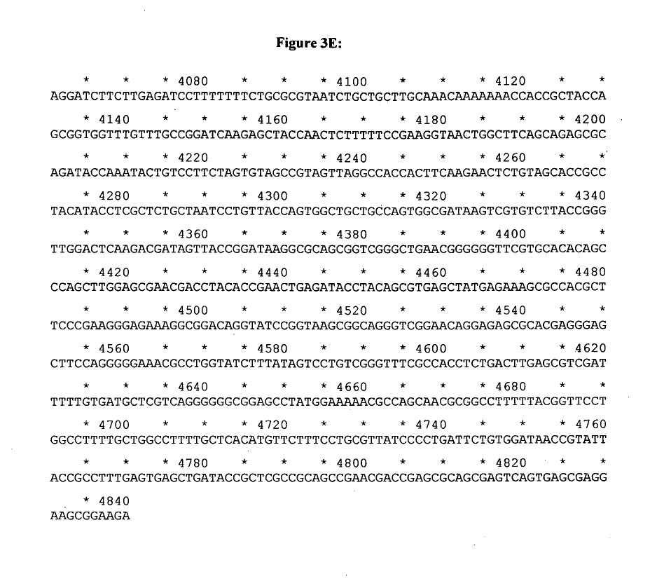

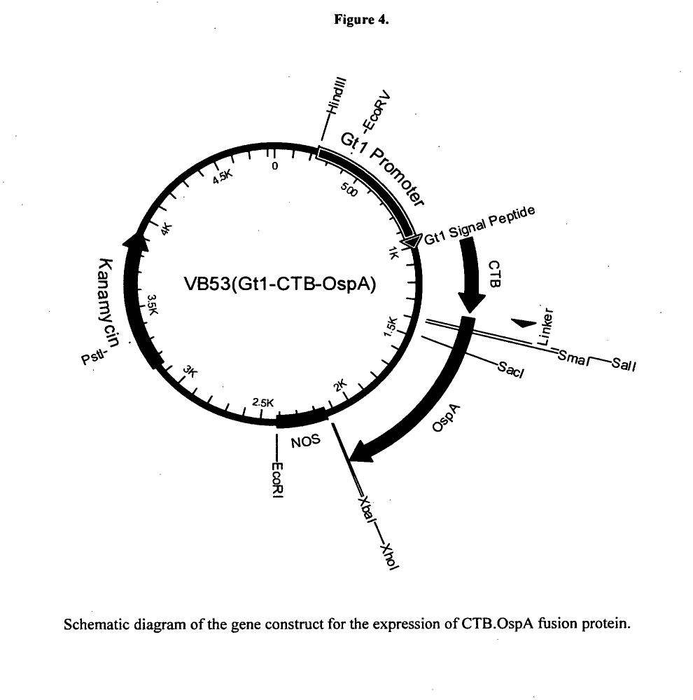

In previous studies, even though high levels of LA2 equivalent antibody in mouse serum was observed and mice were protected when rice-derived OspA is needle inoculated, similar LA2 equivalent antibody titers were not observed in mouse serum when rice-derived OspA was fed orally. A chimeric protein consisting of antigen and adjuvant was therefore created to more specifically target the mucosa of the intestine. If antigens are linked with the B subunit of cholera toxin (CTB), antibody titers may be induced earlier during the immunization schedule and at a higher level. Thus, a CTB.OspA fusion protein was designed to be expressed in rice grain. To develop a CTB.OspA fusion gene construct, the codon-optimized OspA gene (SwissProt P14013) was modified for codon-optimization. In the newly codon-optimized OpsA gene sequence, 84% (216 out of 257) of codons were altered, and the G+C content was increased to 62% from 34% in the native OspA nucleotide sequence. The amino acid sequence of CTB (P01556) was also back-translated into a nucleotide sequence with the codons biased towards rice codon usage preference. In the codon optimized CTB gene sequence, 84% (87 out of 103) of codons were modified, and G+C content was increased to 61.8% from 34.3% in the native CTB gene nucleotide sequence. CTB.OspA fusion protein sequence with an intervening linker of six amino acid residues (PGPGPG; identified herein as SEQ ID NO: 3) was back-translated into a nucleotide sequence with codons biased to rice proteome. The codon-optimized CTB.OspA fusion gene sequence was synthesized by Integrated DNA Technology (IDT) (SEQ ID NO: 1) with Mly I and a Xho I restriction sites engineered at 5′ and 3′ ends of the codon-optimized CTB.OspA gene, and cloned into plasmid pIDTSMART to create the plasmid “pIDTSMART-AMP:CTOS” (CTB.OspA). The codon-optimized OspA and CTB.OspA genes were re-verified by creating translation maps with DS Gene program. The plasmid DNAs containing the synthesized CTB.OspA gene were transformed into NEB 10 E. coli cells, and the plasmid DNA pIDTSMART-AMP:CTOS (CTB.OspA) was renamed “VB52;” The CTB.OspA insert fragment was released with MlyI+XhoI from plasmid VB52, and then ligated in frame into NaeI/XhoI-digested pAPI405 vector, which contains the rice seed storage protein glutelin 1 gene (GenBank accession no. Y00687) promoter (Gt1), signal peptide encoding sequence, and the terminator of the nopaline synthase (nos) gene of the T-DNA in Agrobacterium tumefaciens. The resultant plasmid is designated as “VB53” (FIGS. 3A-3E and 4; SEQ ID NO: 3).

SEQ ID NO: 1 represents a codon-optimized CTB.OspA
fusion nucleic acid sequence:
ACCCCGCAGAACATCACCGACCTCTGCGCGGAGTACCACAACACCCAGAT
CCACACCCTCAACGACAAGATCTTCTCCTACACCGAGAGCCTGGCCGGCA
AGCGCGAGATGGCGATCATCACCTTCAAGAACGGCGCCACCTTCCAGGTC
GAGGTGCCGGGCTCCCAGCACATCGACAGCCAGAAGAAGGCCATCGAGCG
CATGAAGGACACCCTCCGCATCGCCTACCTCACCGAGGCCAAGGTCGAGA
AGCTCTGCGTCTGGAACAACAAGACCCCGCACGCCATCGCCGCCATCTCC
ATGGCCAACCCCGGACCAGGGCCGGGGTGCAAGCAGAACGTCAGCTCCCT
GGACGAGAAGAACTCCGTCAGCGTCGACCTCCCGGGCGAGATGAAGGTGC
TCGTGTCCAAGGAGAAGAACAAGGACGGGAAGTACGACCTCATCGCCACC
GTGGACAAGCTGGAGCTCAAGGGCACCTCCGACAAGAACAACGGGTCCGG
CGTCCTGGAGGGGGTGAAGGCGGACAAGAGCAAGGTCAAGCTCACCATCT
CCGACGACCTCGGCCAGACCACGCTGGAGGTCTTCAAGGAGGACGGCAAG
ACCCTCGTCTCCAAGAAGGTGACCTCCAAGGACAAGTCCAGCACCGAGGA
GAAGTTCAACGAGAAGGGCGAGGTGAGCGAGAAGATCATTACCCGCGCGG
ACGGCACCCGCCTGGAGTACACCGGCATCAAGTCCGACGGCTCCGGGAAG
GCCAAGGAGGTGCTGAAGGGCTACGTGCTGGAGGGGACCCTGACCGCGGA
GAAGACCACCCTGGTGGTCAAGGAGGGCACCGTGACCCTCAGCAAGAACA
TCGCGAAGTCCGGCGAGGTGTCCGTCGAGCTGAACGACGCCGACAGCTCC
GCCGCGACCAAGAAGACCGCGGCCTGGAACTCCGGGACCTCCACCCTCAC
CATCACCGTCAACAGCAAGAAGACGAAGGACCTCGTGTTCACGAAGGAGA
ACACGATCACCGTGCAGCAGTACGACAGCGCCGGCACCAAGCTGGAGGGC
AGCGCGGTGGAGATCACCAAGCTCGACGAGATCAAGAACGCGCTCAAGTG
ATAG
SEQ ID NO: 2 represents the amino acid sequence of
a CTB.OspA fusion protein:
TPQNITDLCAEYHNTQIHTLNDKIFSYTESLAGKREMAIITFKNGATFQV
EVPGSQHIDSQKKAIERMKDTLRIAYLTEAKVEKLCVWNNKTPHAIAAIS
MANPGPGPGCKQNVSSLDEKNSVSVDLPGEMKVLVSKEKNKDGKYDLIAT
VDKLELKGTSDKNNGSGVLEGVKADKSKVKLTISDDLGQTTLEVFKEDGK
TLVSKKVTSKDKSSTEEKFNEKGEVSEKIITRADGTRLEYTGIKSDGSGK
AKEVLKGYVLEGTLTAEKTTLVVKEGTVTLSKNIAKSGEVSVELNDADSS
AATKKTAAWNSGTSTLTITVNSKKTKDLVFTKENTITVQQYDSAGTKLEG
SAVEITKLDEIKNALK

### Example 8

### Plant Transformation

FIG. 4 diagrams the VB53 plasmid construct for the expression of CTB.OspA fusion protein. The construct includes a promoter from the rice glutelin (Gt1) seed storage protein; a region encoding the Gt1 signal peptide (SP); a region encoding the Cholera toxin B subunit protein (CTB); a region encoding a six amino acid alternating proline-glycine peptide linker; a region encoding outer surface protein A (OspA) of B. burgdorferi; and a nopaline synthase (Nos) gene terminator from A. tumefaciens.

Gene Transformation—

The linear expression cassette of DNA fragments comprising the region from promoter to terminator (without the backbone plasmid sequence) of VB53 plasmid was liberated with EcoRI and HindIII double digestion and used for microprojectile bombardment-mediated transformation of embryonic calli induced from the mature seeds of cultivar Bengal (Oryza sativa, subsp. Japonica). A total of 146 independent transgenic events from the transformation with plasmid VB53 were shown to contain the fusion transgene via PCR, and were cultured in the greenhouse.

Identification and Selection of Genetically Stable Transgenic Lines Expressing CTB.OspA

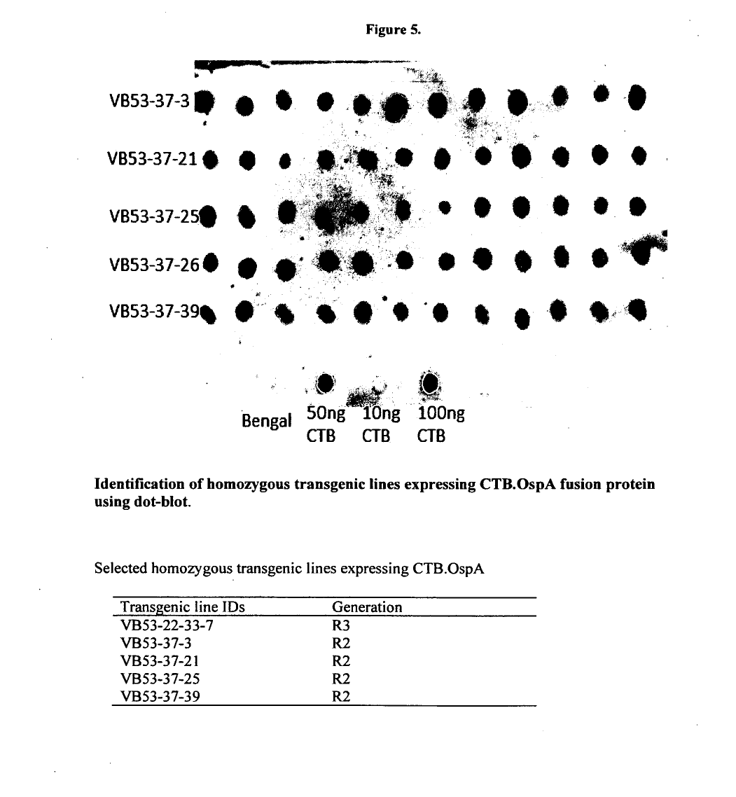

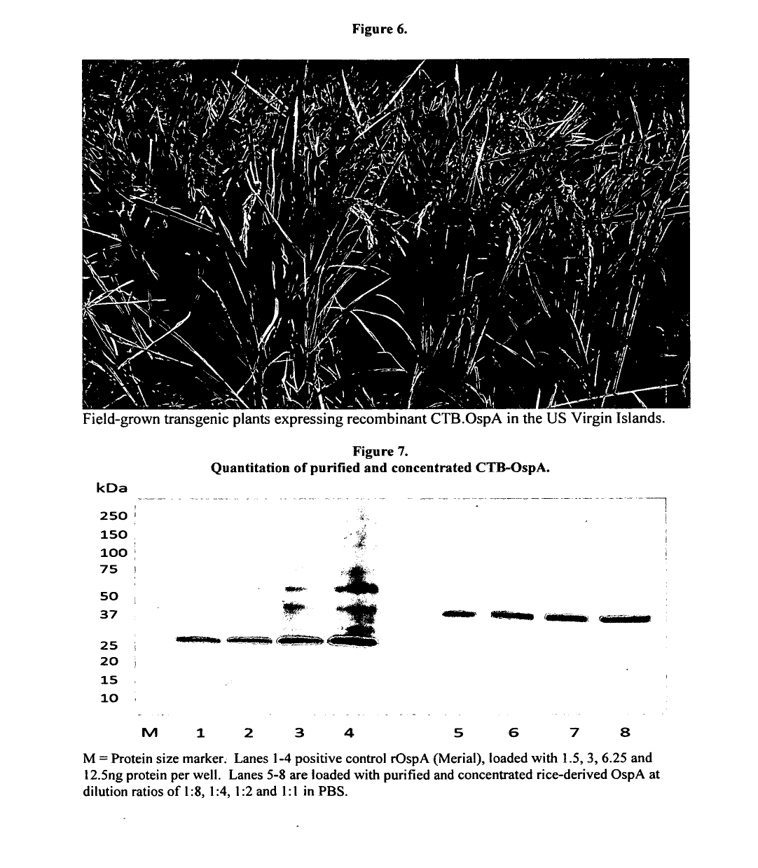

By using an immune dot blot with anti-CTB or anti-OspA antibodies (FIG. 5), four homozygous transgenic lines were identified as expressing CTB.OspA. Total soluble seed proteins were extracted with 0.25 ml of PBS buffer, pH 7.4 per seed at room temperature for 20 min followed by centrifugation. 3 μl of protein extract from 12 seeds of a transgenic line were spotted onto a nitrocellulose membrane. The blot was probed with anti-CTB antibody. “Bengal” indicates the non-transgenic rice cultivar; different transgenic lines are indicated by the labels “VB53-37-3,” “VB53-37-21,” “VB53-37-25,” “VB53-37-26” and “VB53-37-39;” “CTB” indicates E. coli-derived recombinant CTB (Sigma). After expression screening of transgenic R1 seeds, two positive transgenic events, VB53-22 and VB53-37, were selected to grow out to subsequent generations for selection of homozygous lines. The line VB53-22-33-7 had been grown in a US Virgin Islands nursery site in 2011/2012 winter season for scale-up seed production (FIG. 6), and grown in a field production site in Junction City, Kans. in May 2012 along with the other four homozygous lines to produce seeds for large scale protein purification.

### Example 9

### Development of Protocol to Purify CTB.OspA Protein

Purification of CTB.OspA from Transgenic Rice Seeds.

A preliminary purification protocol for CTB.OspA protein was developed using seeds available from the early generation harvest from the US Virgin Islands site. Following milling of rice seed into flour, proteins were extracted with extraction buffer (25 mM sodium phosphate [NaPi], 50 nM NaCl, pH 6.0) at a buffer: flour ratio of 5:1. The extract was clarified by passing it through a CellPure filter aid and subsequently through 0.2 μm filtration units (Millipore). The protein filtrates were then loaded onto a DEAE chromatography column equilibrated in 25 mM NaPi, 50 nM NaCl, pH 6.0. The CTB.OspA fusion protein did not bind to this column and was recovered in in the column flow-through. The partially purified CTB.OspA was loaded onto a SP Sepharose column equilibrated in 25 mM NaPi, 50 nM NaCl, pH 6.0. CTB.OspA bound to the SP column and remained so during a wash with 25 mM NaPi containing 200 mM NaCl. The CTB.OspA fraction was eluted with a high-ionic strength buffer consisting of 25 mM NaPi, 500 mM NaCl, pH 6.0. Purified CTB.OspA was concentrated and exchanged into PBS by tangential flow filtration (Millipore Pellicon, 10 kDa filter unit). Analysis of purified CTB.OspA by SDS-PAGE demonstrated a prominent band at approximately 39 kDa, where the fusion protein was predicted to migrate.

The yield of rice CTB.OspA was estimated by immunoblotting. Commercially available rOspA (Merial) was used as a standard in a Western blot assay and LA-2 antibody to detect the protein. Proteins were prepared in SDS-sample buffer containing 5% β-mercaptoethanol, denatured by boiling at 90° C. for 5 minutes, resolved on a 4-20% Tris-glycine SDS-PAGE gel, and then blotted and probed with LA-2 antibody. FIG. 7 demonstrates the ability of LA-2 antibody to react with both Merial vaccine OspA and rice CTB.OspA fusion protein. Merial OspA migrates at approximately 28 kDa, whereas the fusion protein disclosed herein migrates at a higher banding position of approximately 39 kDa. The purified and concentrated CTB.OspA fusion protein was estimated to be approximately 10 μg/ml by this method.

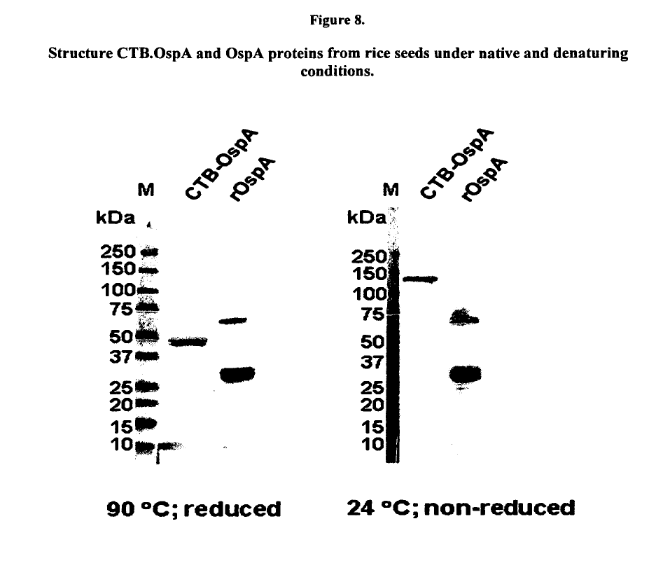

In order to further characterize the OspA-CTB fusion protein, and to determine whether there was inherent polymeric organization, as is seen with native CTB which forms pentamers under native conditions, Western blotting assays were conducted under reducing and non-reducing conditions using anti-OspA antibody. Proteins were either reduced and heated at 90° C. for 5 min or non-reduced and non-heated, then resolved on a 4-20% Tris-glycine SDS-PAGE gel, and blotted and probed with anti-OspA monoclonal antibody H5332 for immunodetection. As shown in FIG. 8, the Western blot revealed a predominant immune band at about 150 kDa, suggesting the formation of a multimeric complex. However, when run under denaturing and reducing conditions, bands were only evident at approximately 39 kDa, suggesting the presence of monomeric forms of CTB.OspA only. In a separate Western blot probed with anti-CTB antibody, the presence of free CTB was proved in rice grains, which most likely resulted from the partial breaking down of CTB.OspA fusion protein (data not shown). Without being bound by theory, it is believed that the particular multimeric complex of 150 kDa in FIG. 7 is composed of three molecules of CTB.OspA (3×40 kDa=120 kDa) and two molecules of CTB (2×12 kDa=24 kDa), and that this particular pentameric molecule is functional, while other combinations are not.

### Example 10

### Development of GM1 Binding ELISA to Determine Maintenance of CTB Function

To further characterize and quantify the CTB.OspA fusion protein, an ELISA based on the ability of native CTB to bind the GM1 receptor was developed, and CTB.OspA fusion protein to react with the protective LA-2 antibody. For this assay, commercially available GM1 (Sigma) was used to coat a 96-well Immulon microwell plate. GM1 was solubilized to 1 mg/mL in dimethylformamide. 100 μl GM1 (final concentration 10 μg/ml in ELISA coating buffer [90 mM NaHCO3, 60 mM Na2CO3. pH 9.6]) was added to each well and incubated at 4° C. overnight. The plate was then washed five times in TBT-T buffer (10 mM Tris, 140 mM NaCl, 2.7 mM KCl, 0.05% Tween 20, pH 7.4) and blocked with 250 μl blocking buffer (TBS-T, 3% BSA) for 2 hr at 37° C. Defined standards (rOspA, MerialCTB.OspA, CTB [Sigma]) were prepared by serial 2-fold dilution from 800 ng 1000 ng/mL-1.5 ng/mL and rice-derived CTB.OspA was prepared in TBS-T. The concentration range of CTB.OspA was estimated as 10 μg/mL from Western blot, as described previously. Standards or samples (100 μL) were added to each well and incubated at 37° C. for 1 hr, and subsequently washed 5× in TBS-T. Antibodies directed against either CTB (Abcam #34992) or LA-2 (1:2,000 dilution) were then applied to predetermined wells containing either standard or sample, and incubated for 1 hr at 37° C. Following another wash step, alkaline phosphatase-labeled secondary antibody (1:10,000) was applied to each well and incubated at 37° C. for lhr. Following an additional wash step, 100 μl p-nitrophenyl phosphate disodium salt (pNPP, 1 mg/ml in diethanolamine, Thermo Fisher) was added to each well and incubated for 20 min at room temperature. To stop the reaction, 50 μL of 2N NaOH was added to each well and absorbance was determined at 405 nm.

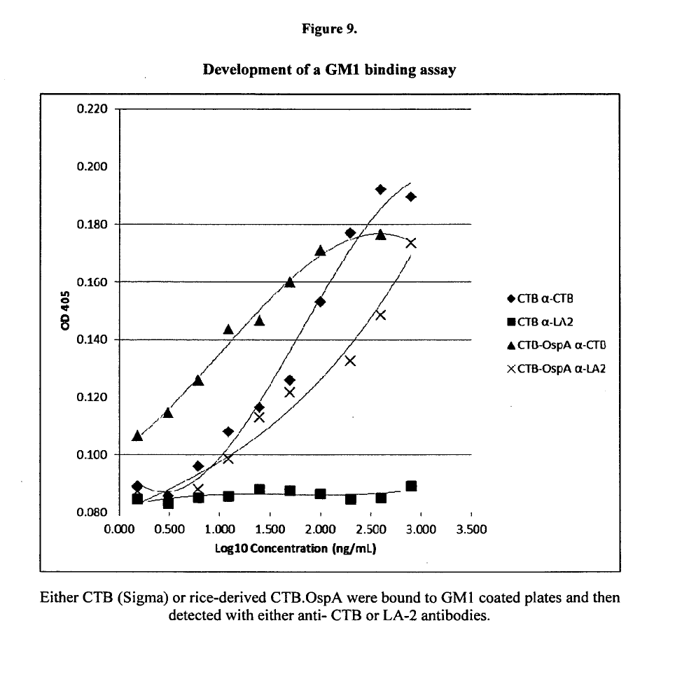

FIG. 9 demonstrates similar binding characteristics between CTB and CTB.OspA when anti-CTB was used as a detection antibody. However, the curve of CTB.OspA is shifted to the right when LA-2 is used as the detection antibody. Previous estimations of protein concentration by Western blot are consistent with the results of this assay when using anti-CTB as a detection antibody. In addition, this assay demonstrates the preservation of both CTB and the protective moiety of the CTB.OspA fusion protein.

Together, these data demonstrate the successful generation of a recombinant CTB.OspA fusion protein that i) migrates at the predicted size in a SDS-PAGE gel, ii) exhibits appropriate structural conformations due to its ability to recognize anti-CTB and anti-OspA antibodies, and iii) forms multimeric complexes under native conditions in a predictable stoichiometric ratio.

### Example 0.11

### CTB.OspA is Immunogenic and Induces Neutralizing Antibodies when Orally Administered to Mice

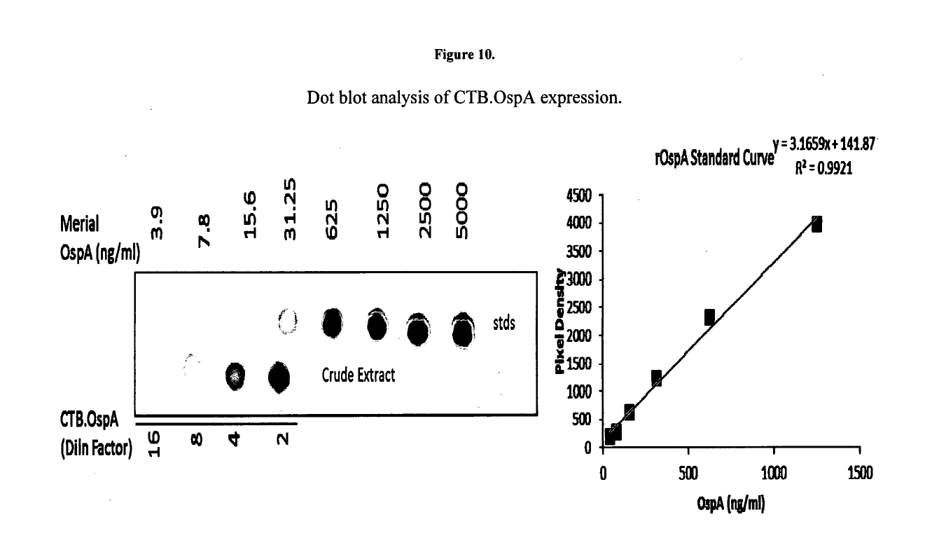

Preparation of bait. In order to assess the amount of CTB.OspA being consumed by the mice, a dot blot assay was used to determine the CTB.OspA concentration against known standards of E. coli derived OspA (Rekombitek, Merial). CTB.OspA flour was mixed with phosphate buffered saline (PBS) at a flour:buffer ratio of 1:5 for 30 mins at room temperature. The soluble protein extract was clarified by passing it through a CellPure filter aid and Whatmann filter paper. For example, soluble protein was extracted from 40 g CTB.OspA flour in 200 ml buffer and filtered with CellPure filteraid through a Whatmann membrane. A dilution series of CTB.OspA extract was prepared by twofold serial dilution into PBS to ensure the detected signal would fall on the linear standard curve. 3 μl of extract or standard where blotted onto a nitrocellulose membrane. The membrane was then blocked with 3% nonfat dry milk in TBS-tween 0.05% for 30 mins, followed by incubation with LA-2 antibody (1 μg/ml) for 1 hr and then probed with goat anti-rabbit HRP (40 ng/ml, Pierce) for 30 mins. Estimates of protein concentration were made by comparing against a standard curve using merial rOspA. The membrane was then developed with Supersignal West Femto (Pierce) reagent and resultant chemiluminescence measured with FlourChem Q CCD camera system (Protein Simple). It was thus determined that each gram of transgenic flour contained 4.81±1.78 μg CTB.OspA protein (FIG. 10). With the previous observation that mice consume 4 g of flour bait daily (FIG. 1), it was estimated that the amount of vaccine to be consumed by the mice was roughly 20 ug.

To increase that amount of antigen being consumed by the mice, the rice flour was enriched with lyophilized extract. To achieve this, 400 g soluble protein was extracted in 2 litres of PBS for 30 min. The slurry was then centrifuged at 9,000×g for 30 mins and the supernatant lyophilized. The lyophilized product was then mixed with 200 g CTB.OspA flour and a dough was made by adding 100 mls water. The dough was then rolled into cylindrical shape and cut into sections of roughly 1 inch and 3 g in weight. The resultant baits were allowed to dry overnight in a laminar flow hood at room temperature.

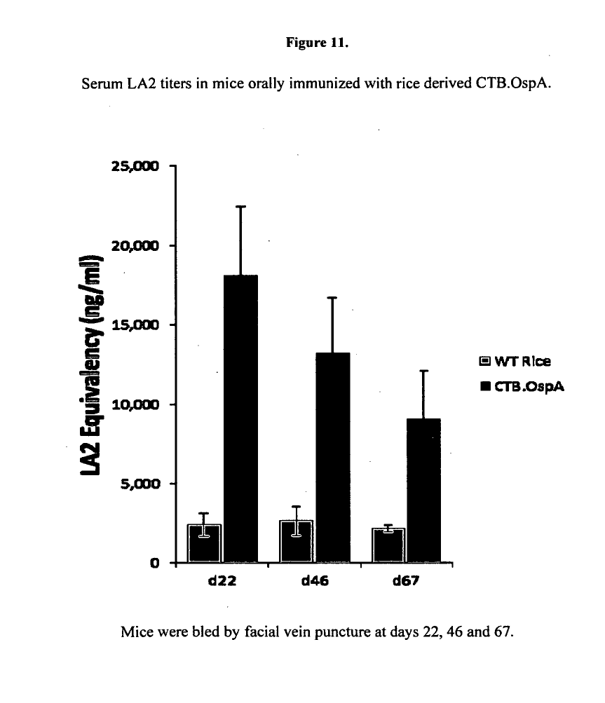

Female C3H/HeJ mice (four weeks) were purchased from Jackson Labs. Oral immunization was initiated when the mice were six weeks old. Seven mice were assigned to feeding continually with bait comprised of CTB.OspA flour. Five mice were fed on bait made with wild type flour. Blood samples for serum preparation were collected by facial artery/vein plexus bleeding at 22, 46 and 67 days after initiating vaccine feeding. LA-2 titers were determined by ELISA. FIG. 11 demonstrates an elevated serum LA-2 response as early as 22 days after feeding on CTB.OspA bait. However, the antibody titers were observed to decrease in a time dependent manner over the duration of the experiment. The reason for this may be due to the generation of immunological tolerance, or due to unknown dietary factors resulting from a limited diet of rice flour over an extended period of time.

Using data derived from previous experiments, it was determined that the latter serum LA-2 levels were not likely to either protect the mice from B. burgdorferi infected ticks, or to clear pre-existing infection in ticks feeding on the immunized mice. Therefore, the dose of delivery of CTB.OspA will be increased in the baits in an effort to determine a dose response, and to modify the feeding schedule such that increased concentration of antigen may be administered over fewer doses in an effort to reduce the likelihood of development of oral tolerance.

### Example 12

### Laboratory Mice Protected by Immunization with Injected Rice-Derived OspA and Clearance of B. burgdorferi Infection in Vector Ticks

Immunization of C3H/HeJ Mice with Purified Rice-Derived OspA.—

This animal study was conducted under protocol 09-007, which was approved by the CDC DVBID Institutional Animal Care and Use Committee (IACUC). C3H/HeJ mice, a strain that is highly susceptible to B. burgdorferi infection, were used for all immunizations. The immunogens were OspA purified from recombinant rice, and a positive control of E. coli-derived rOspA from a commercially available source (Recombitek Lyme, Merial). PBS formulated with adjuvant served as a negative control throughout the study. The antigens were diluted to specified concentrations in PBS and delivered subcutaneously in a 1:1 emulsified formula with Freund's Complete Adjuvant (CFA). 3 groups of mice (n=6) were treated with rice-derived OspA at a concentration of 25, 50 or 75 μg/dose, delivered in a volume of 50 μl. A primary immunization was administered at day 0. Two booster immunizations were administered with Incomplete Freund's Adjuvant (ICFA, 1:1) on days 21 and 42. In order to track the generation of the protective IgG antibody, mice were bled at the facial vein plexus using a 5 mm lancet while the mice were under isoflurane anaesthesia at days 35 and 57 and serum analyzed by ELISA. In order to detect the presence of a previously described anti-OspA IgG antibody, a competition-based ELISA was modified exploiting the use of the LA-2 monoclonal antibody raised against OspA. Following observation of high LA-2 equivalent IgG antibody titers, mice were exposed to infected ticks which were allowed to feed until repletion. Fed ticks were crushed, placed in culture, and monitored for the presence or absence of B. burgdorferi by dark field microscopy over a period of 4 weeks. In order to assess the infection of mice, tissue samples (blood, skin, kidney and heart biopsies) were harvested upon termination of the experiment and cultured for the detection of B. burgdorferi. The ability of mice to produce anti-OspA antibodies, and the determination of infection was further carried out by immunoblot against whole-cell antigen of B. burgdorferi strain B31 (Viramed Biotech AG, Germany). This demonstrated that recombinant OspA purified from transgenic rice is highly immunogenic, and protective against both transmission of B. burgdorferi to mice, and also is adequate for clearance of infection in feeding ticks.

Measurement of LA-2 Equivalent IgG Antibody Titers in Serum of Immunized Mice.

Following primary vaccination and two boosting immunizations of C3H/HeJ mice with rOspA, mice were bled at the facial vein plexus and serum was prepared by allowing the blood to clot for 12 h at 4° C., followed by centrifugation at 5,000×g. Serum samples were diluted 1:800 and analyzed for their ability to compete with biotinylated LA-2 antibody in a semi-competitive ELISA, modified from known protocols (See Johnson et al., (1995) Vaccine 13:1086-1095). In brief, ELISA plates were coated with 100 μl of 100 ng/ml commercially available recombinant OspA (Merial) diluted in bicarbonate/carbonate coating buffer (90 mM NaHCO₃, 60 mM Na₂CO₃. pH 9.6) and incubated overnight at 4° C. After washing 5 times with TBS-T, each well was blocked with 250 μl TBS-T containing 1% bovine serum albumin, and incubated at 37° C. for lhr. Following an additional wash step, 1000, diluted samples/standards were applied to each well and incubated for 1 hr at 37° C. After a subsequent wash step, 100 μl biotinylated LA-2 antibody (100 ng/ml) was added to each well and the plate was incubated at 37° C. for 1 hr. Following a final washing step, 100 μl peroxidase-labeled streptavidin (1 μg/ml) was then added to each well and incubated at 37° C. for lhr. Following a final wash step, 100 μl of a peroxidase substrate solution (SureBlue Reserve TMB, KPL) was added to each well and incubated at room temperature for 20 min. 100 μl stop solution was then added to each well (TMB blueSTOP, KPL) and the absorbance was measured at 320 nm. The modification of the LA-2 competitive ELISA protocol allowed for both a broader dynamic range and an increase in sensitivity. Following serum evaluation at d57, it was demonstrated that the antibody titer was above the threshold required for the clearance of B. burgdorferi in infected feeding ticks (Table 8). Furthermore, the elevated antibody titer was evident upon the termination of the experiment 16 weeks after the initial immunizations were administered.

Table 8 shows serum LA-2-equivalence titers of C3H/HeJ mice injected with various doses of rice-derived rOspA, Merial rOspA or sham-treated. “NA” means that the sample was unavailable due to mortality of the mouse.

TABLE 8

| | | α-LA-2 | | α-LA-2 | |
|---|---|---|---|---|---|
| | | (μg/ml) | Avg | (μg/ml) | Avg. |
| Group | ID | Day 57 | (±SEM) | Day 113 | (±SEM) |
| Rice rOspA | 6961 | 459 | 347 (±48) | NA | 455 (±30) |
| 20 μg | 6962 | 372 | | 496 | |
| | 6963 | 217 | | 375 | |
| | 6964 | 239 | | 531 | |
| | 6965 | 505 | | 398 | |
| | 6966 | 287 | | 479 | |
| Rice rOspA | 6967 | 164 | 411 (±64) | 103 | 418 (±75) |
| 50 μg | 6968 | 335 | | 212 | |
| | 6969 | 405 | | 363 | |
| | 6970 | 624 | | 585 | |
| | 6971 | 523 | | 537 | |
| | 6972 | 416 | | 394 | |
| Rice rOspA | 6973 | 252 | 459 (±110) | 144 | 418 (±50) |
| 75 μg | 6974 | 287 | | 196 | |
| | 6975 | 296 | | 185 | |
| | 6976 | 973 | | 389 | |
| | 6977 | 489 | | 444 | |
| | 6978 | 455 | | 236 | |
| Merial | 6956 | 419 | 1358 (±402) | 460 | 1668 (±526) |
| | 6957 | 2487 | | 3222 | |
| | 6958 | 993 | | 869 | |
| | 6959 | 765 | | 1216 | |
| | 6960 | 2127 | | 2587 | |
| PBS | 6951 | 0 | 0 | 0 | 0 |
| | 6952 | 0 | | 0 | |
| | 6953 | 0 | | 0 | |
| | 6954 | NA | | NA | |
| | 6955 | 0 | | 0 | |

LA-2 antibody titers were increased in all treatment groups receiving either rice-derived rOspA or E. coli-derived rOspA compared with sham-immunized controls, which exhibited undetectable levels of LA2 antibody. No observable dose-dependent effect was observed with rice-derived rOspA, suggesting an efficacy of treatment at as little as 20 μg rOspA/dose.

Feeding efficiency of Ixodes scapularis nymphs on rOspA immunized mice. All mice were challenged with colony-raised I. scapularis nymphs infected with B. burgdorferi strain B31. Five B31− infected nymphs were placed on the necks of each immunized and control mouse, whilst the mice were under isoflurane anaesthesia. The average number of ticks feeding to repletion was 4±0.89.

Protection of Mice and Clearance of B. burgdorferi Infection in Fed Ticks.

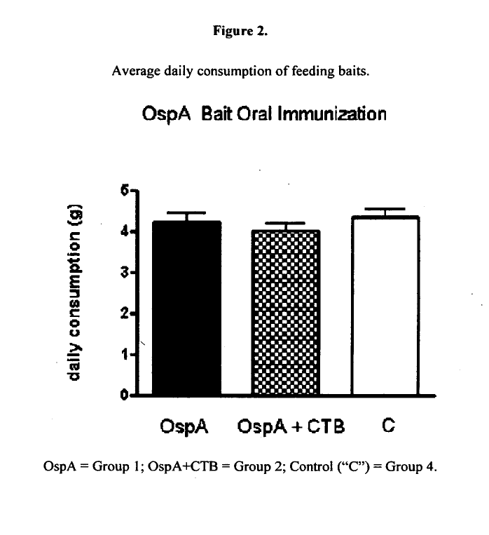

All animals receiving rOspA developed high levels of LA-2 equivalent IgG antibody (Table 9). Table 9 also demonstrates protection of all animals immunized with rice-derived rOspA. The tick challenges were effective since all PBS control animals became infected with B. burgdorferi. Protection was assessed in two ways, by culture of tissues and by serology. Cultures of tissues from all three body sites (skin, heart, and bladder) were uniformly negative. Serum was analyzed by immunoblots (Viramed, Inc.) for evidence of antibody reaction with whole cell antigens extracted from cultured Borrelia (especially OspC and FlaB) and with recombinant VIsE, an antigen upregulated in expression during B. burgdorferi infection of mammals. Rice OspA-immunized mice did not develop antibodies to OspC, FlaB, VlsE or any other antigens characteristic of B. burgdorferi infection (FIG. 2). In contrast, serum from animals injected with PBS alone reacted with numerous diagnostic Borrelia antigens.

Of 64 nymphal ticks recovered after feeding on mice, only a few five were infected. One tick remained infected in both the 20 μg and the 75 μg rice OspA groups, and three were documented in the 50 μg group. These “break-through” infections were not correlated with LA-2 antibody levels in the mice on which they fed. In the PBS control group, 19/20 replete ticks remained infected. The lack of infection in one replete tick in this group is not unexpected, since the rate of B. burgdorferi (“Bb”) infection in the tick colony used for challenges is not 100%.

A trend toward increased mean LA-2 equivalent antibody titers was observed with increasing dose of rice OspA antigen (347, 411, and 459 μg/ml, respectively). The lowest observed LA-2 equivalent titer, however, was sufficient both to protect mice and clear ticks. The minimum LA-2 equivalent level for a successful vaccine is unknown to date. Mice were protected from infected ticks when LA-2 equivalent antibody levels are 4 μg/ml. This low level did not clear ticks of infection. The antibody level necessary to clear ticks might be estimated from the work of de Silva et al. (1999) Infect. Immun. 67:30-35, although that group used a different protective monoclonal antibody and method, and determined that clearing ticks requires about 36 times as much protective antibody as protecting mice (213 μg/ml in de Silva's system, and projected to be about 144 μg/ml in the present system). In view of this, an LA-2 equivalent titer of 120 μg/ml achieved by an oral immunization protocol is considered sufficiently high to warrant challenging mice with infected ticks.

Table 9 shows the efficacy of rice rOspA in protecting mice and clearing B. burgdorferi from infected ticks. (“NA” means that the sample was unavailable due to mortality).

TABLE 9

| | | α-LA-2 titer | Bb +/total | Skin | Heart | Bladder |
|---|---|---|---|---|---|---|
| Group | ID | (μg/ml) | replete ticks | biopsy | biopsy | biopsy |
| Rice | 6961 | 459 | NA | NA | NA | NA |
| rOspA | 6962 | 372 | 0/4 | − | − | − |
| 20 μg | 6963 | 217 | 1/2 | − | − | − |
| | 6964 | 239 | 0/5 | − | − | − |
| | 6965 | 505 | 0/2 | − | − | − |
| | 6966 | 287 | 0/5 | − | − | − |
| Rice | 6967 | 164 | 0/3 | − | − | − |
| rOspA | 6968 | 335 | 1/4 | − | − | − |
| 50 μg | 6969 | 405 | 1/5 | − | − | − |
| | 6970 | 624 | 0/4 | − | − | − |
| | 6971 | 523 | 1/4 | − | − | − |
| | 6972 | 416 | 0/3 | − | − | − |
| Rice | 6973 | 252 | 1/5 | − | − | − |
| rOspA | 6974 | 287 | 0/4 | − | − | − |
| 75 μg | 6975 | 296 | 0/3 | − | − | − |
| | 6976 | 973 | 0/4 | − | − | − |
| | 6977 | 489 | 0/4 | − | − | − |
| | 6978 | 455 | 0/4 | − | − | − |
| Merial | 6956 | 419 | 0/4 | − | − | − |
| | 6957 | 2487 | 0/4 | − | − | − |
| | 6958 | 993 | 0/5 | − | − | − |
| | 6959 | 765 | 0/3 | − | − | − |
| | 6960 | 2127 | 0/4 | − | − | − |
| PBS | 6951 | 0 | 4/5 | + | + | + |
| | 6952 | 0 | 5/5 | + | + | + |
| | 6953 | 0 | 5/5 | + | + | + |
| | 6954 | NA | NA | NA | NA | NA |
| | 6955 | 0 | 5/5 | + | + | + |

When the LA2 antibody in mouse serum reaches 9000 ng/ml, mice will be challenged with infected nymphal ticks to see if mice can be protected and spirochetes cleared from the midguts of ticks.

The needle immunization study via subcutaneous administration is being repeated with several concentrations of transgenic rOspA and the mice challenged directly with infected nymphal ticks. It is being determined whether the correct epitope(s) of OspA are presented at levels in vivo to induce resistance to tick-transmitted spirochetes. Threshold LA2 levels needed for future oral immunization studies will be set.

Previous studies used 12.5 μg of purified OspA per immunization. After two boosts, the LA2 equivalent antibody level, on average, was 8805 ng/ml with a range from 1,386 to 34,378 ng/ml. In ongoing studies, immunization groups receiving 20 μg, 30 μg and 75 μg of purified rOspA will be tested, administering three doses subcutaneously with booster immunizations occurring at 2 and 4 weeks. Seven days after the last immunization, mice will be challenged with infected nymphal ticks. Mice and fed ticks will then be analyzed for B. burgdorferi infection as described earlier.

Adjuvant Effect of Rice-Derived CTB:

It will be determined if rice-derived CTB is effective in boosting immunogenicity of rice-derived OspA. Based on experiments carried out with rice-derived OspA, the most effective dose of OspA will be used. Rice flour containing the most effective dose of OspA will be added with rice flour containing differing amounts of CTB, ranging from 25 μg to 200 μg/dose. The mixture of OspA and CTB flour will be made into feeding bait for oral immunization. Blood will be collected every three weeks from experimental mice and LA2 antibody will be measured to examine the effect of CTB in boosting immune response of OspA. A shorter immunization time is predicted when the appropriate amount of CTB is delivered in OspA flour.

Mice with high levels of LA2 antibody along with appropriate controls will then be challenged with infected nymphal ticks to determine if mice are indeed protected and sprirochetes cleared from the midgut of ticks.

Expression Analysis and Homozygous Line Selection of CTB-OspA Fusion:

R1 seeds will be harvested to identify the plants expressing a CTB-OspA fusion protein. Events that express high levels of CTB-OspA fusion will be selected and planted to recover a R2 generation. Seeds will then be harvested and dot blot analysis will be done as described for rCTB production. As described supra, analysis will be carried out to identify homozygous events and expression stability. Homozygous lines will then be selected and this new generation of rice will be planted for bulk seed production to generate a sufficient amount of the seeds for animal studies. While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A monocot plant-expressed fusion protein comprising cholera toxin B subunit (CTB) adjuvant fused to outer surface protein A (OspA) derived from a Borrelia species, or fragment thereof.

2. A formulation for oral administration to an animal, comprising a CTB.OspA fusion protein having at least 90% sequence identity to the sequence identified by SEQ ID NO: 2.

3. A chimeric gene for expression of a CTB.OspA fusion protein, comprising:
(i) a promoter that is active in monocot plant cells; and
(ii) operably linked to the promoter, a nucleic acid sequence having at least 90% sequence identity to SEQ ID NO: 1, encoding an amino acid sequence expressing a fusion protein comprising cholera toxin B subunit (CTB) adjuvant fused to an outer surface protein A (OspA) protein or fragment thereof.

4. The amino acid sequence (SEQ ID NO: 2) encoded by the nucleic acid sequence of claim 3.

5. (canceled)

6. A transgenic monocot plant expressing a CTB.OspA fusion protein having at least 90% sequence identity to the sequence identified by SEQ ID NO: 2.

7. A rice seed product comprising the fusion protein expressed by the chimeric gene of claim 3.

8-10: (canceled)

11. A method of immunizing an animal against infection with a Borrelia species pathogen, comprising the step of administering the CTB.OspA fusion protein to said animal.

12. The method of claim 11, wherein the formulation is administered orally in an amount effective to induce the production of specific antibodies to the OspA, wherein said antibodies are effective to ameliorate or clear infection by a Borrelia species pathogen in mammals.

13. The method of claim 11, wherein the formulation is orally administered in an amount from about 1 microgram to about 100 mg of the at least one CTB.OspA fusion protein per day.

14. An oral vaccine produced by
a) providing a transgenic plant cell expressing the chimeric gene of claim 3,
b) producing a plant from the transgenic plant cell and growing it for a time sufficient to produce seeds containing the CTB.OspA fusion protein,
c) harvesting mature seeds containing the CTB.OspA fusion protein,
d) grinding the mature seeds into small particles,
e) optionally extracting the CTB.OspA fusion protein from the seeds,
f) optionally producing a flour from the mature seeds, and
g) optionally combining the CTB.OspA with one or more excipients.

15. The oral vaccine of claim 14, comprising at least one CTB.OspA fusion protein and, optionally, one or more excipients formulated for oral administration.

16. The oral vaccine of claim 15, wherein said vaccine is provided in a form selected from the group consisting of a bait, pellet, tablet, caplet, hard capsule, soft capsule, lozenge, cachet, powder, granules, suspension, solution, elixir, liquid, beverage, and food.

17. A method of breaking a Lyme disease cycle by controlling pathogen prevalence in reservoir animals, comprising the steps of:
a) expressing a CTB.OspA fusion protein having at least 90% sequence identity to the sequence identified by SEQ ID NO: 2 in monocot seeds;
b) producing a rice flour from the monocot seeds;
c) formulating the rice flour into a reservoir-targeting oral vaccine formulation without extracting the CTB.OspA fusion protein from the seeds; and
d) administering the formulation to Lyme disease reservoirs to induce immunity in reservoir species, thus reducing pathogen levels in reservoir animals and associated vectors.

18. (canceled)

19. A method of producing an CTB.OspA fusion protein in monocot plants, comprising the steps of:
(a) transforming a monocot plant cell with the chimeric gene of claim 3;
(b) producing a monocot plant from the transformed monocot plant cell and growing it for a time sufficient to produce seeds containing the CTB.OspA; and
(c) harvesting the seeds from the monocot plant.

20-29. (canceled)

30. The formulation of claim 2, wherein the fusion protein is admixed with an antibiotic.

31. The method of claim 11, wherein the administering comprises administering the formulation of claim 2 to said animal.

32. The method of claim 11, wherein the administering comprises administering the rice seed product of claim 7 to said animal.


SEQUENCE LISTING (3 sequences)

SEQ ID NO: 1 - DNA, 1104 bp, Artificial Sequence
  Recombinant DNA sequence
accccgcagaacatcaccgacctctgcgcggagtaccacaacacccagatccacaccctcaacgacaagatcttctcctacaccgagagcctggccggcaagcgcgagatggcgatcatcaccttcaagaacggcgccaccttccaggtcgaggtgccgggctcccagcacatcgacagccagaagaaggccatcgagcgcatgaaggacaccctccgcatcgcctacctcaccgaggccaaggtcgagaagctctgcgtctggaacaacaagaccccgcacgccatcgccgccatctccatggccaaccccggaccagggccggggtgcaagcagaacgtcagctccctggacgagaagaactccgtcagcgtcgacctcccgggcgagatgaaggtgctcgtgtccaaggagaagaacaaggacgggaagtacgacctcatcgccaccgtggacaagctggagctcaagggcacctccgacaagaacaacgggtccggcgtcctggagggggtgaaggcggacaagagcaaggtcaagctcaccatctccgacgacctcggccagaccacgctggaggtcttcaaggaggacggcaagaccctcgtctccaagaaggtgacctccaaggacaagtccagcaccgaggagaagttcaacgagaagggcgaggtgagcgagaagatcattacccgcgcggacggcacccgcctggagtacaccggcatcaagtccgacggctccgggaaggccaaggaggtgctgaagggctacgtgctggaggggaccctgaccgcggagaagaccaccctggtggtcaaggagggcaccgtgaccctcagcaagaacatcgcgaagtccggcgaggtgtccgtcgagctgaacgacgccgacagctccgccgcgaccaagaagaccgcggcctggaactccgggacctccaccctcaccatcaccgtcaacagcaagaagacgaaggacctcgtgttcacgaaggagaacacgatcaccgtgcagcagtacgacagcgccggcaccaagctggagggcagcgcggtggagatcaccaagctcgacgagatcaagaacgcgctcaagtgatag

SEQ ID NO: 2 - protein, 366 aa, Artificial Sequence
  Recombinantly expressed protein
TPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANPGPGPGCKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNIAKSGEVSVELNDADSSAATKKTAAWNSGTSTLTITVNSKKTKDLVFTKENTITVQQYDSAGTKLEGSAVEITKLDEIKNALK

SEQ ID NO: 3 - DNA, 5058 bp, Artificial Sequence
  Recombinant DNA sequence
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaagttcagctatgaccatgattacgccaagcttcatgagtaatgtgtgagcattatgggaccacgaaataaaaaggttaacattttgatgagtcgtgtatcctcgatgagcctcaaaagttctctcaccccggataagaaacccttaacgttgcaatgtgcaaagtttgcattctccactgacataatgcaaaataagatatcatcgatgacatagcaactcgttatgcatcatatcatgcctctctcaacctattcattcctactcatctacataagtatcttcagctaaatgtgtttagaacataaacccataagtcacgtttgatgagtattaggcgtgacacatgacaaatcacagactcaagcgttaagataaagcaaaatgatgtgtacataaaactccagagctatatgtcatattgcaaaaagaggagagcttgttataagacaaggcatgactcacaaaaattcatttgcctttcgtgtcaaaaagaggagggctttacattatcgttcatgtcatattgcaaaagaaagagagagagaacaacacaatgctgcgtcaattatacatatctgtatgtcgttcatcattattcatccacctttcgtgtaccacacttcatatatcatgagtcacttcatgtctggacattaagttcaaactctatcttaacatttagatgcaagagcctttatctcactataaatgcacgatgatttctcattgtgtgatagttttctcacaaaaagcggccgcttcattagtcctacaacaacatggcatccataaatcgccccatagttttcctttcgtgattccgagttcacagtttgcttgttcctcttgtgcgatggctccctagccaccccgcagaacatcaccgacctctgcgctatttagcggagtaccacaacacccagatccacaccctcaacgacaagatcttctcctacaccgagagcctggccggctatgatgcaagcgcgagatggcgatcatcaccttcaagaacggcgccaccttccaggtcgaggtgccgggctcccagctatatcacatcgacagccagaagaaggccatcgagcgcatgaaggacaccctccgcatcgcctacctcaccgaggctactaaaaccaaggtcgagaagctctgcgtctggaacaacaagaccccgcacgccatcgccgccatctccatggccaagggctaacccccggaccagggccggggtgcaagcagaacgtcagctccctggacgagaagaactccgtcagcgtcgacaaggactcccgggcgagatgaaggtgctcgtgtccaaggagaagaacaaggacgggaagtacgacctcatcgccaacatgtgggccgtggacaagctggagctcaagggcacctccgacaagaacaacgggtccggcgtcctggagggggtgaaaatgttggcggacaagagcaaggtcaagctcaccatctccgacgacctcggccagaccacgctggaggtcttcaagagtttgaggacggcaagaccctcgtctccaagaaggtgacctccaaggacaagtccagcaccgaggagaagttcaagtagttgacgagaagggcgaggtgagcgagaagatcattacccgcgcggacggcacccgcctggagtacaccggcataggaggttacaagtccgacggctccgggaaggccaaggaggtgctgaagggctacgtgctggaggggaccctgaccgcggcattgttaggagaagaccaccctggtggtcaaggagggcaccgtgaccctcagcaagaacatcgcgaagtccggcgaggaaaattaagttgtccgtcgagctgaacgacgccgacagctccgccgcgaccaagaagaccgcggcctggaactccgggacattttttctccaccctcaccatcaccgtcaacagcaagaagacgaaggacctcgtgttcacgaaggagaacacgatcatagtgataccgtgcagcagtacgacagcgccggcaccaagctggagggcagcgcggtggagatcaccaagctcgacgaaaagatcaagaacgcgctcaagtgatagctcgagtctagacgcgtcccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatccgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatagcattcaaatatgtatccgctcatgagacaataaccctgataaatgcttcaataatattgcaagcgaaccggaattgccagctggggcgccctctggtaaggttgggaagccctgcaaagtaaactggatggctttcttgcaaccgccaaggatctgatggcgcaggggatcaagatctgatcaagagacaggatgaggatcgtttcgcatgataactgaacaagatggattgcacgcaggttctccggccgcttgggtggagaggctattcggctatgactgggcaaaccaacagacaatcggctgctctgatgccgccgtgttccggctgtcagcgcaggggcgcccggttctttttgtaactcaagaccgacctgtccggtgccctgaatgaactgcaggacgaggcagcgcggctatcgtggctggccacaacgacgggcgttccttgcgcagctgtgctcgacgttgtcactgaagcgggaagggactggctgctattgggcaacgaagtgccggggcaggatctcctgtcatctcgccttgctcctgccgagaaagtatccatcatggctgatgaaccaatgcggcggctgcatacgcttgatccggctacctgcccattcgaccaccaagcgaaacatcgcatcgaaacgcgagcacgtactcggatggaagccggtcttgtcgatcaggatgatctggacgaagagcatcaggggctcaacgcgccagccgaactgttcgccaggctcaaggcgcgcatgcccgacggcgaggatctcgtcgtgatccatgaacgcgatgcctgcttgccgaatatcatggtggaaaatggccgcttttctggattcaacgactgtggctggctaacgggtgtggcggaccgctatcaggacatagcgttggatacccgtgatattgctgaagagcttggcggcgaaaactgggctgaccgcttcctcgtgctttacggtatcgccgctcccgattcgcagcgcatcgccttctatcgccaacttcttgacgagttcttctgaattgaaaaaggaagagtttaaaaggatctaggtgaagatcctttttgataatctcatgaccaaaatcccttaacgtgagttttcgttccactgagcgtcagaccccgtagaaaagatcaagaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga